SEQUENCE LISTING (28 sequences)

SEQ ID NO: 1 - DNA, 26 bp, West Nile virus
acgctgccacaagtcctggttccctg

SEQ ID NO: 2 - DNA, 26 bp, West Nile virus
cctcccaaacatgtagagtctcacat

SEQ ID NO: 3 - DNA, 26 bp, West Nile virus
ccaaattgccgcagactcgttgtgaa

SEQ ID NO: 4 - DNA, 26 bp, Dengue virus type 2
tcacattcagatatgttggttcccac

SEQ ID NO: 5 - DNA, 26 bp, Dengue virus type 2
taaatgtgacgttcacagacaagtcc

SEQ ID NO: 6 - DNA, 26 bp, Dengue virus type 2
gctgatacactgaagtgttctgattg

SEQ ID NO: 7 - DNA, 24 bp, Artificial
  Biotin_F- Forward primer for priming out signal peptide with NheI cut site
ctagctagctccggcctgaacgac

SEQ ID NO: 8 - DNA, 32 bp, Artificial
  Biotin_WDIII_F- Forward primer for overlapping signal peptide and WNV DIII protein
gacgacgacaagagcctgaagggaacatatgg

SEQ ID NO: 9 - DNA, 27 bp, Artificial
  Biotin_WDIII_R- Reverse primer for overlapping signal peptide and WNV DIII protein
tgttcccttcaggctcttgtcgtcgtc

SEQ ID NO: 10 - DNA, 30 bp, Artificial
  WDIII_R- Reverse primer for priming out WNV DIII protein from WNV cDNA
ccgctcgagttagctcccagatttgtgcca

SEQ ID NO: 11 - DNA, 94 bp, Artificial
  BN-B03-DNA- Biotinylated DNA sequence corresponding to the B03 synthesized for comparison studies
gaaggtgaaggtcggctgaagcattagacctaagcacgctgccacaagtcctggttccctggcttaggtctaatgcaccatcatcaccatcttc

SEQ ID NO: 12 - DNA, 94 bp, Artificial
  BN-B99-DNA- Biotinylated DNA sequence corresponding to the B99 synthesized for comparison studies
gaaggtgaaggtcggctgaagcatcagacctaagcccaaattgccgcagactcgttgtgaagcttaggtctaatgcaccatcatcaccatcttc

SEQ ID NO: 13 - DNA, 24 bp, Artificial
  DENV1 BN-rEDIII Primers for Bacterial expression Primer A (BAP Forward)
ctagctagctccggcctgaacgac

SEQ ID NO: 14 - DNA, 33 bp, Artificial
  DENV1 BN-rEDIII Primers for Bacterial expression Primer B (BAP Reverse)
atatgacatcccttttaagctcttgtcgtcgtc

SEQ ID NO: 15 - DNA, 33 bp, Artificial
  DENV1 BN-rEDIII Primers for Bacterial expression Primer C (D1-DIII Forward)
gacgacgacaagagcttaaaagggatgtcatat

SEQ ID NO: 16 - DNA, 27 bp, Artificial
  DENV1 BN-rEDIII Primers for Bacterial expression Primer D (D1-DIII Reverse)
ccgctcgagttagcttcccttcttgaa

SEQ ID NO: 17 - DNA, 24 bp, Artificial
  DENV2 BN-rEDIII Primers for Bacterial expression Primer A (BAP Forward)
ctagctagctccggcctgaacgac

SEQ ID NO: 18 - DNA, 33 bp, Artificial
  DENV2 BN-rEDIII Primers for Bacterial expressionPrimer B (BAP Reverse)
gtatgacattcctttgaggctcttgtcgtcgtc

SEQ ID NO: 19 - DNA, 33 bp, Artificial
  DENV2 BN-rEDIII Primers for Bacterial expression Primer C (D2-DIII Forward)
gacgacgacaagagcctcaaaggaatgtcatac

SEQ ID NO: 20 - DNA, 24 bp, Artificial
  DENV2 BN-rEDIII Primers for Bacterial expression Primer D (D2-DIII Reverse)
ccgctcgagttaacttcctttctt

SEQ ID NO: 21 - DNA, 24 bp, Artificial
  DENV3 BN-rEDIII Primers for Bacterial expression Primer A (BAP Forward)
ctagctagctccggcctgaacgac

SEQ ID NO: 22 - DNA, 33 bp, Artificial
  DENV3 BN-rEDIII Primers for Bacterial expression Primer B (BAP Reverse)
atagctcatccccttgaggctcttgtcgtcgtc

SEQ ID NO: 23 - DNA, 33 bp, Artificial
  DENV3 BN-rEDIII Primers for Bacterial expression Primer C (D3-DIII Forward)
gacgacgacaagagcctcaaggggatgagctat

SEQ ID NO: 24 - DNA, 27 bp, Artificial
  DENV3 BN-rEDIII Primers for Bacterial expression Primer D (D3-DIII Reverse)
ccgctcgagttagctccccttcttgta

SEQ ID NO: 25 - DNA, 24 bp, Artificial
  DENV4 BN-rEDIII Primers for Bacterial expression Primer A (BAP Forward)
ctagctagctccggcctgaacgac

SEQ ID NO: 26 - DNA, 33 bp, Artificial
  DENV4 BN-rEDIII Primers for Bacterial expression Primer B (BAP Reverse)
gtatgacattcccttgatgctcttgtcgtcgtc

SEQ ID NO: 27 - DNA, 33 bp, Artificial
  DENV4 BN-rEDIII Primers for Bacterial expression Primer C (D4-DIIIForward)
gacgacgacaagagcatcaagggaatgtcatac

SEQ ID NO: 28 - DNA, 27 bp, Artificial
  DENV4 BN-rEDIII Primers for Bacterial expression Primer D (D4-DIII Reverse)
ccgctcgagttaactccctttcctgaa

(19) United States
(12) Patent Application Publication
(10) Pub. No.: US 2017/0226511 A1
(43) Pub. Date: Aug. 10, 2017

(54) APTAMERS FOR BINDING FLAVIVIRUS PROTEINS

(71) Applicant: NATIONAL UNIVERSITY OF SINGAPORE, Singapore (SG)

(72) Inventors: Mah Lee Mary NG, Singapore (SG); Krupakar PARTHASARATHY, Singapore (SG); Jin Shun Anthony CHUA, Singapore (SG); Hui Yu Haydan YEO, Singapore (SG)

(73) Assignee: National University of Singapore, Singapore (SG)

(21) Appl. No.: 15/036,451

(22) PCT Filed: Nov. 13, 2014
(86) PCT No.: PCT/SG2014/000532

§ 371 Date: May 13, 2016

(30) Foreign Application Priority Data
Nov. 13, 2013 (SG) 201308420-7

(51) Int. Cl. C12N 15/115; A61K 45/06; G01N 33/569; A61K 31/713

(52) U.S. Cl. CPC C12N 15/115; A61K 31/713; A61K 45/06; G01N 33/56983; C12N 2310/16; C12N 2310/3513; C12N 2310/3517; C12N 2320/31; G01N 2333/185

(57) ABSTRACT

The present invention relates to nucleic acids. In particular, it relates to aptamers capable of binding to a flavivirus structural protein or a flavivirus non-structural protein, useful as therapeutics for preventing, treating and/or diagnosing a flavivirus infection in a patient.

### FIELD OF THE INVENTION

The present invention relates to nucleic acids. In particular, it relates to aptamers capable of binding to a flavivirus structural protein or a flavivirus non-structural protein, useful as therapeutics for preventing, treating and/or diagnosing a flavivirus infection in a patient.

### BACKGROUND OF THE INVENTION

The Flaviviridae family is composed of seventy enveloped positive single-stranded. RNA viruses. Of the seventy, several are clinically relevant human pathogens, which include Dengue virus (DENV), yellow fever virus (YFV), Japanese encephalitis virus (JEV), West Nile virus (WNV) and tick-borne encephalitis virus (TBEV) (Chavez et al., 2010, Noda et al., 2012). Besides Flavivirus, the Flaviviridae family consists of two other genera, Pestivirus and Hepacivirus (Chavez et al., 2010). Flaviviruses are mostly arboviruses and are transmitted to hosts via infected mosquitoes. The virions of flaviviruses are usually small, in the form of an enveloped particle with a diameter of 40-60 nm. Flaviviruses, specifically Dengue and West Nile have resulted in a wide divergent of diseases with no available vaccines or antiviral specific drugs for human treatment to date (Chavez et al., 2010).

West Nile virus (WNV), a flavivirus (Saxena et al., 2013, Bigham et al., 2011) transmitted by mosquitoes, is a member of the Japanese encephalitis virus (JEV) sero-group within the Flaviviridae family. The other members include Cacipacore virus, Murray Valley encephalitis virus and St. Louis encephalitis virus. Kunjin virus found in Australia and Asia is also a subtype of WNV. WNV was first isolated in 1937 from a woman in the West Nile region of Uganda (Silva et al., 2013, Duan et al., 2009) and was first reported in New York City in 1999 (Silva et al., 2013). WNV is a neurotropic flavivirus and is capable of causing neurological diseases in human, horses and some bird species (Silva et al., 2013). Its genome is a positive single-stranded RNA that is 11,029 nucleotides long and the virions are small, spherical, enveloped, and approximately 50 nm in diameter (Bigham et al., 2011). The most common symptoms of WNV are fever, headache, and/or hepatitis. A recent WNV outbreak in 2012 in the United States reported 5387 cases and 243 deaths (CDC report) (Saxena et al., 2013). No approved vaccine or treatment in human is available to date (CDC report) (Duan et al., 2009). The genomic and proteomic organizations of WNV are very similar to those of Dengue virus. Dengue virus (DENY), a mosquito-borne viral pathogen, is a member of the Flaviviridae family. DENV consists of four serotypes (DENV1, DENV2, DENV3 and DENV4). DENV has a positive-sense, 11-kb RNA genome that contains both structural and non-structural proteins in a single polyprotein (Gromowski et al., 2007, Crill et al., 2001, Lisova et al., 2007, Rajamanonmani et al., 2009). The gene order is C-prM-E-NS1-NS2A-NS2B-NS3-NS4A-NS4B-NS5. The viral envelope consists of lipid bilayers where envelope (E) and membrane (M) proteins are embedded. The E protein is 495 amino acids in length and is glycosylated in DENV as well as in WNV. In particular, its N-linked glycosylation at Asn-67 is essential for virus propagation and is unique to DENV (Rey, 2003). The functional roles of E protein are its involvement in virus attachment to cells and also in membrane fusion (Clyde et al., 2006, Modis et al., 2004). It has also been demonstrated to be highly immunogenic and is able to elicit production of neutralizing antibodies against wild-type virus. The dengue E protein comprises of 3 regions: Domain-I (DI), Domain-II (DII) and Domain-III (DIII). DI is the central domain; DII is the dimerization and fusion domain, while DIII is an immunoglobulin-like receptor binding domain (Mukhopadhyay et al., 2005, Rey et al., 1995). It has been proven that DIII domain is a receptor recognition and binding domain (Bhardwaj et al., 2001, Chin et al., 2007, Chu et al., 2005, Zhang et al., 2007). Thus DIII is an important target for therapeutic development against DENV. Infected humans can manifest symptoms that vary from being asymptomatic, to a febrile disease, to a potentially fatal internal hemorrhage (Teoh et al., 2012, Noda et al., 2012), Immunity against different dengue serotypes are mediated by serotype-specific antibodies. Hence, patients who have recovered from the infecting serotype are thought to have perennial immunity towards the infecting serotype but short-lived immunity against other serotypes (Teoh et al., 2012). As reported by the Centre for Disease Control and Prevention, there are as many as one hundred million people infected yearly (CDC report). A recent report cautioned that the global distribution of dengue infection might even exceed 390 million per year (Bhatt et al., 2013). To date, no approved vaccine or antiviral therapeutic is available in the clinical market for humans (Teoh et al., 2012).

One way of detecting the WNV and DENV is through the use of antibodies. However, the use of antibody detection has been shown to be non-specific and engineering or inserting a novel detection moiety is difficult.

Therefore, there is a need in the art for alternative methods for detecting, treating and preventing flavivirus infections in patients.

### SUMMARY OF THE INVENTION

The present invention relates to aptamers capable of binding to a flavivirus structural protein or a flavivirus non-structural protein. Such apatamers are useful as therapeutics for preventing, treating and/or diagnosing a flavivirus infection in a patient. Like antibodies, aptamers are able to bind to the surface of viruses. However, the advantage of aptamers over antibodies is the possibility of the introduction of chemically engineered detection moieties to aptamers. Also, the production cost for aptamers is lower than antibodies, as aptamers are synthesized chemically. Aptamers are also easy to customize, stable, no requirement for cold transport chain and have higher binding affinities to antigens as compared to antibodies.

In a first aspect of the invention, there is provided a nucleic acid aptamer comprising a DNA molecule that binds specifically to a flavivirus structural protein or a flavivirus non-structural protein.

Preferably, the flavivirus is selected from the group consisting of West Nile virus, Dengue virus, yellow fever virus, Japanese encephalitis, and tick-borne encephalitis virus.

In a preferred embodiment, the aptamer binds specifically to a West Nile virus envelope protein, and preferably the aptamer binds specifically to the Domain III region of the West Nile virus envelope protein. In this embodiment, the DNA molecule is preferably a modified DNA molecule based on one of three native aptamer sequences: (a) the sequence of the West Nile Virus envelope protein DIII 5′-ACGCTGCCACAAGTCCTGGTTCCCTG-3′ (SEQ ID NO: 1); (b) the sequence of the West Nile Virus envelope protein DIII 5′-CCTCCCAAACATGTAGAGTCTCACAT-3′ (SEQ ID No: 2); or (c) the sequence of the West Nile Virus envelope protein DIII 5′-CCAAATTGCCGCAGACTCGTTGTGAA-3′ (SEQ ID NO: 3) and comprising amino acid side chains. Preferably, the modified DNA molecule comprises a sequence selected from the group consisting of:
(a) 5′-A_CfGkC_T_GwChC_A_CfAlA_GbT_ChC_T_GwGbT_T_CyChC_T_Gw-3′ (based on modification of SEQ ID No. 1) or its complement;
(b) 5′-ChC_T_CyChC_AlA_A_CfAeT_GbT_AsG_AsG_T_CyT_CyA_CfAeT-3′ (based on modification of SEQ ID No. 2) or its complement; and
(c) 5′-ChC_AlA_AeT_T_GwChC_GkC_AsG_A_CfT_CyGbT_T_GwT_GwAlA_-3′ (based on modification of SEQ ID No. 3) or its complement,
   wherein functional groups of side chains are indicated in lowercase (b: Thiophene, e: Glutamic acid, f: Phenylalanine, h: Histidine, k: Lysine, l: Leucine, s: Serine, y: Tyrosine, w: Tryptophan) and native nucleotides are indicated with an underscore (_).

In an alternative preferred embodiment, the aptamer binds specifically to a Dengue virus envelope protein, and preferably the aptamer binds specifically to the Domain III region of the Dengue virus envelope protein. In this embodiment, the DNA molecule is preferably a modified DNA molecule based on one of three native aptamer sequences: (a) the sequence of DENV 2 envelope protein DIII TCACATTCAGATATGTTGGTTCCCAC-3′(SEQ ID NO: 4); (b) the sequence of DENV 2 envelope protein DIII 5′-AAATGTGACGTTCACAGACAAGTCC-3″ (SEQ ID No: 5); or (c) the sequence of DENV 2 envelope protein DIII 5′-GATACACTGAAGTGTTCTGATTG-3′ (SEQ ID NO: 6) and comprising amino acid side chains. Preferably the modified DNA molecule comprises a sequence selected from the group consisting of:
(a) 5′ T-CyA_CfAeT_T_CyAsG_AeT_AeT_GbT_T_GwGbT_T_CyChC_A_Cf-3′ (based on modification of SEQ ID No. 4) or its complement;
(b) 5′-T_AkAlA_T_GwT_GwA_CfGbT_T_CyA_CfAsG_A_CfAlA_GbT_ChC_-3′ (based on modification of SEQ ID No. 5) or its complement; and
(c) 5′-GkC_T_GwAeT_A_CfA_CfT_GwAlA_GbT_GbT_T_CyT_GwAeT_T_Gw-3′ (based on modification of SEQ ID No. 6) or its complement,
   wherein functional groups of side chains are indicated in lowercase (b: Thiophene, e: Glutamic acid, f: Phenylalanine, h: Histidine, k: Lysine, l: Leucine, s: Serine, y: Tyrosine, w: Tryptophan) and native nucleotides are indicated with an underscore (_).

In both embodiments, the DNA molecule may further comprise a detectable moiety. The detectable moiety may be selected from the group consisting of biotin, enzymes, chromophores, fluorescent molecules, chemiluminescent molecules, phosphorescent molecules, coloured particles and luminescent molecules. Preferably, the detectable moiety is biotin.

Preferably, the aptamer further comprises a drug of interest, wherein the binding of the DNA molecule to a flavivirus structural or non-structural protein targets the drug of interest to its intended site of action and/or releases the drug of interest from the aptamer. Preferably, the drug is selected from the group consisting of a pharmaceutical compound, a nucleotide, an antigen, a steroid, a vitamin, a hapten, a metabolite, a peptide, a protein, a peptidomimetic compound, an imaging agent, an anti-inflammatory agent, a cytokine, and an immunoglobulin molecule or fragment thereof.

Methods of attaching various agents or drugs to antibodies or aptamers and other target site-delivery agents are well known in the art, and so methods of preparing aptamers of the invention comprising a drug of interest will be readily apparent to the person skilled in the art.

The drug or agent may be chemically or biologically conjugated to the aptamer of the invention. In particular, any method for conjugating a drug or agent to a DNA molecule also can be used. However, it is recognized that, regardless of which method of producing a conjugate of the invention is selected, a determination must be made that the DNA molecule maintains its targeting ability and that the drug maintains its relevant function.

The drug or agent may be released from the aptamer after the binding of the aptamer to its specific target. The release of the drug or agent may be by any method known to the skilled person. For example, the drug or agent may be cleaved by the host by way of a trigger molecule or mechanism. Alternatively, the drug or agent may be released by photo-activation. Radiation for the release of the drug in its active form can be provided by one of a variety of means, depending upon the photo sensitivities of the chosen photolabile bond, the DNA molecule and the drug. This may comprise the use of electromagnetic radiation, for example infrared, visible or ultraviolet radiation, supplied from incandescent sources, natural sources, lasers including solid state lasers or even sunlight.

In a second aspect of the invention, there is provided an aptamer according to the first aspect of the invention for use in diagnosis. Preferably, the aptamer of the invention is used in diagnosis of a flavivirus infection in a patient. The patient is preferably human but may be any animal, mammal, primate or the like.

In a third aspect of the invention, there is provided an aptamer according to the first aspect of the invention for use in therapy. Preferably the aptamer of the invention is used in the treatment or prevention of flavivirus infection in a patient.

In a fourth aspect of the invention, there is provided an immunogenic composition or vaccine comprising an aptamer according to the first aspect of the invention. Generally, a vaccine refers to a therapeutic material, treated to lose its virulence and containing antigens derived from one or more pathogenic organisms, which on administration to a patient, will stimulate active immunity and protect against infection with these or related organisms, whilst an immunogenic composition refers to any pharmaceutical composition containing an antigen, for example, a microorganism, or a component thereof, which composition can be used to elicit an immune response in a patient.

In a fifth aspect of the invention, there is provided a composition comprising an aptamer according to the first aspect of the invention and an excipient or carrier. Pharmaceutically-acceptable excipient may be, for example, antiadherents, binders, coatings, disintegrants, flavours, colours, lubricants, gildants, sorbents, preservatives and sweeteners. An example of a pharmaceutically-acceptable carrier is a carrier protein which facilitates the diffusion of different molecules across a biological membrane.

In a sixth aspect of the invention, there is provided a kit comprising an aptamer according to the first aspect of the invention and a carrier. Preferably, the carrier may be biodegradable nano-particles containing chemotherapeutic agents, photo-agents or quantum dots. The carrier may be conjugated with the aptamer for use in diagnostic/therapeutic applications or therapeutics development. Also, preferably, the kit is used for detecting a flavivirus infection in a patient.

In a seventh aspect of the invention, there is provided an ex vivo method for diagnosing or detecting a flavivirus infection in a patient, the method comprising: (a) obtaining a biological sample from a patient; (b) contacting the biological sample with an aptamer according to the first aspect of the invention; (c) detecting the formation of the binding complex between the aptamer and a flavivirus structural protein and/or a flavivirus non-structural protein, wherein the presence of the binding complex indicates that the patient has a flavivirus infection. The step of detecting the formation of the binding complex may be carried out by conjugating an agent or drug chemically or biologically to the aptamer of the invention. The SELEX (Systematic Evolution of Ligands by Exponential Enrichment) procedure may be used to obtain high affinity and highly specific aptamers against the target protein. The major advantage of the aptamer is that the value of the dissociation constant (K_{D}) towards the target protein lies in the nanomolar ranges. The sequence with the high affinity is taken and conjugated with the biotin molecule which may be detected by streptavidin HRP (horseradish peroxidase).

Preferably, the flavivirus is selected from the group consisting of West Nile virus, Dengue virus, yellow fever virus, Japanese encephalitis and tick-borne encephalitis virus.

Preferably, the biological sample is a blood sample, saliva or urine. As used herein, the term “blood sample” includes blood cells, serum and plasma. More preferably, the biological sample is a blood sample.

In an eighth aspect of the invention, there is provided a method for treating or inducing an immune response to a flavivirus infection in a patient, the method comprising administering to the patient a therapeutically effective dose of the composition or vaccine according to the fifth and sixth aspects of the invention. The mode of administration may be by way of intravenous, oral, pulmonary, ocular, parental, depot or topical. Preferably, the mode of administration is intravenous.

### BRIEF DESCRIPTION OF THE FIGURES

FIG. 1: Cloning strategies. A. Schematic diagram showing the overlapping extension PCR (OE-PCR) technique to obtain biotinylated West Nile Envelope protein domain III (WNE-BNrDIII) for the screening and evaluation of aptamers. Fragment A is designed such that its 3′ overhang is complementary to the 5′ overhang of Fragment B. As such, primer B and primer C are complementary to each other. Both fragments are joined together via the complementary sequence and primers A and D. B. Construct of recombinant WNE-BNrDIII protein generated using OE-PCR. Biotin acceptor peptide (BAP) is downstream of the 6×His tag and thrombin cleavage site, but upstream of the enterokinase cleavage site, whereas the other fragment encodes for the WNE-rDIII protein. 6×His tag at the N-terminal is used for affinity purification while BAP is the signal peptide for biotinylation. Thrombin and enterokinase cleavage sites are included to obtain protein-of-interest without tags. C. Schematic representation of the construct showing the affinity tag and the protein of interest.

FIG. 2: Production of purified biotinylated WNE-BNrDIII protein. (A)(i). SDS-PAGE analysis for expression of the recombinant protein in E. coli BL21 DE3. Lane 2 shows the lysate from uninduced cells and lanes 3 and 4 show the lysate from cells induced using 1 mM IPTG. The expressed recombinant protein is indicated by an asterisk. (A)(ii) Western blot for the expressed recombinant protein using anti-His antibody. The protein construct consists of a 6×His purification tag, and thus when probed with the anti-His antibody, it appears as a thick band (indicated by an asterisk) in the lysate of the induced cells. (A)(iii) SDS-PAGE profile for nickel-nitrilotriacetic acid (Ni-NTA) metal-affinity chromatography purified BN-WNDIII (FT: Flow-through, W1-W3: Washes, E1-E5: Elutes). The bacterial cells were lysed and the inclusion bodies isolated and purified under denaturing conditions in the presence of 8 M urea. The expected molecular mass ˜15 kDa is indicated by an asterisk. Similar expression and purification conditions were carried out for the nonbiotinylated WNE-rDIII protein. (B) FPLC-SEC chromatography profiles for WNE-BNrDIII. The sample injected is obtained from step-by-step dialysis using reducing urea concentration and also in the presence of detergent Tween-20. Both the traces correspond to UV absorbance of the protein at 280 nm (broken line—Unbiotinylated WN rDIII, continuous line—WNE-BnrDIII. The difference in the sample peak indicates the molecular weight difference between the biotinylated and the non-biotinylated WNDIII protein.

FIG. 3: Schematic representation showing the step-by-step process involved in the production of WNE-rDIII antigen for the screening and evaluation of aptamers.

FIG. 4: Detection of biotinylated and unbiotinylated WNDIII protein. [A(i)]The presence of WNV DIII protein was detected using monoclonal mouse anti-His antibody. Bands can be observed in both unbiotinylated (UB) and biotinylated (B) WNV DIII (lanes 3 and 4). Maltose-binding protein (MBP) does not contain His tag so no band was observed lane 1 and 2.[A(ii)] When the biotinylation was detected directly using streptavidin-HRP secondary antibody, distinct bands can be observed in the biotinylated Lanes (B) of MBP and WNV DIII proteins. (lanes 2 and 4). [B] The presence of biotin in the WNV DIII proteins are detected via ELISA using streptavidin-HRP antibody. Biotinylated proteins (MBP-BN, WDIII-BN) show high absorbance values at 450 nm while unbiotinylated protein (MBP-UBN and WNDIII-UBN) are not detected.

FIG. 5: Peak-top heights of Biacore sensorgram for the different aptamers tested using Surface Plasmon Resonance (SPR). The diamonds marked with numbers 1-10 and the aptamer numbers represented are chosen for further evaluation.

FIG. 6: Enzyme linked modified aptamer sorbent assay (ELMASA) for surface screening. Biotinylated aptamer and biotinylted protein was tested for their binding efficiency in different surfaces like maxisorp, multisorp, Polysorp and medisorp. PolySorp plate has high affinity to molecules of hydrophobic nature. MediSorp has plate surface between PolySorp and MaxiSorp, which allows low background reading with samples containing serum. MaxiSorp plate has high affinity to molecules in a mixture of hydrophilic and hydrophobic molecules. MultiSorp plate has high affinity to molecules of hydrophilic nature. After the coating the aptamer and the protein was probed using the streptavidin HRP followed by incubating with the enzyme substrate for the color development. The absorbance corresponds to the amount from the initial biotinylated aptamer or protein bound to the surface. In this case for biotinylated aptamers, Multisorp plate the absorbance at 450 nm was found to be very low (max abs 0.15), polysorp and medisorp is medium (max abs varied from 2-2.5) and Maxisorp is high (max abs varied from 2.5 to 3) and selected for further evaluation.

FIG. 7: Protein-coated enzyme linked modified aptamer sorbent assay for affinity screening. The West Nile virus envelope DIII protein is coated on the surface, followed by incubation with different concentrations of biotinylated aptamers, and then probing with streptavidin-HRP conjugate. The aptamer which binds strongly to the protein shows high absorbance. B03, B79 and B99 binds to the WNDIII protein as the absorbance is significantly higher when compared to the control and other aptamers (indicated by asterisks).

FIG. 8: West Nile virus-coated enzyme linked modified aptamer sorbent assay for affinity screening. The West Nile virus Wengler strain is coated on the surface and then incubated with different concentrations of biotinylated aptamers, followed by probing with streptavidin-HRP conjugate. The aptamer which binds to the protein shows high absorbance. When compared with the various concentrations of aptamer, aptamers B03, B79 and B99 bind significantly in all the concentrations tested when compared with the control. In contrast, other aptamers only bind to the virus significantly in concentrations higher than 3.3 nM.

FIG. 9: West Nile virus strain Sarafend and Kunjin coated enzyme linked modified aptamer sorbent assay for affinity screening. It was found that aptamers B03, B67, B73 and B99 bind significantly at the concentrations higher than 3.3 nM to the Sarafend strain, while aptamers B03, B66, B67, B73 and B79 bind significantly at the concentrations higher than 3.3 nM to the Kunjin strain.

FIG. 10: Percentage of neutralization for the West Nile virus Wengler strain using 5 μM and 10 μM of modified aptamers.

FIG. 11: Apotox and Alamar blue cell viability assays for the aptamers. BHK cells were grown and treated with different concentrations of aptamers, positive controls (digitonin detergent and MPER-membrane protein extraction reagent) and BSA (as a negative control). The viability was tested at 24, 36, 48 and 60 hours post-treatment. As shown, aptamer treatment at different concentrations (from 3.3 nM to 26 nM) does not alter cell viability when compared with the untreated sample (0 nM). It is evident in the positive control MPER that viability is lost, whereas in the digitonin detergent treated cells, the viability is lost at 24 and 36 hours post-treatment. Interestingly, the cells start to recover at 48 and 60 hours post-treatment. Similar results were obtained using the Alamar blue viability assay.

FIG. 12: Stability assay for aptamers. Top panel (24 hours), Bottom panel (100 hours) incubation. Aptamer bands are detected in gel red after 5 days of incubation at 37° C., indicating that the aptamers are very stable.

FIG. 13: Stability assay for aptamers in serum. Top panel (48 hours), Bottom panel (120 hours) incubation. BN-Aptamer were found to be very stable as detected in ELISA absorbance at 450 nm.

FIG. 14: Schematic representation showing the step by step process involved in the evaluation of modified aptamers against the WNE-rDIII antigen.

FIG. 15: Expression of BAP-WNDIII protein in E. coli BL 21 (DE3) and E. coli K12 strain AVB 100. Left panel. SDS-PAGE analysis for the expression of the recombinant protein in E. coli BL21DE3 (Lane 2 shows lysate from uninduced cells and lanes 3 and 4 show lysates from cells induced using 1 mM IPTG), and E. coli K12 strain AVB 100 (Lane 5 shows lysate from uninduced cells and lanes 6 and 7 show lysates from cells induced using 1 mM arabinose). Expression of the protein was observed in E. coli BL 21(DE3) and not in E. coli K12 strain AVB 100. Right panel. Western blot for the protein expressed in E. coli BL 21 (DE3) and E. coli K12 strain AVB 100 using anti-His antibody. The protein construct consists of a 6×His purification tag, and thus can be identified by a thick band when probed with anti-His antibody, as in the case of E. coli BL 21 (DE3) induced protein (lanes 3 and 4), whereas the bands are absent in the induced E. coli K12 strain AVB 100 (lanes 6 and 7).

FIG. 16: ELISA for determination of in vitro biotinylation using Biotin ligase enzyme. The presence of biotin in WNV DIII protein is detected via ELISA using streptavidin-HRP conjugate. Biotinylated proteins show high absorbance values at 450 nm while unbiotinylated proteins are not detected. High absorbance was observed in both WNDIII-unbiotinylated and also WNDIII in vitro biotinylated proteins using Bir A (sample 1 and sample 2). These results gave us a hint that the BAP-WNDIII protein expressed might be endogenously biotinylated.

FIG. 17: ELISA for the confirmation of biotinylation using Bc-Mac streptavidin magnetic beads. The WNDIII protein was allowed to bind with the streptavidin magnetic beads. If the protein contains biotin it will bind strongly to streptavidin. Elution of the bound protein is done using 0.1 M glycine followed by evaluating the protein by ELISA. Positive control used was biotinylated and non-biotinylated MBP (maltose binding protein). The BAP-WNDIII protein obtained from Bc-Mag bead and also from, the FPLC fraction shows high absorbance, indicating that WNDIII protein was indeed in vivo biotinylated endogenously during expression.

FIG. 18: Evaluation of stability of WNV DIII modified aptamers in human serum. The negative control (B03 heated at 95° C. for 48 hrs) shows a reduced absorbance, indicating that the modified aptamers are not stable at high temperatures. The histograms a, b and c represent modified aptamers incubated in buffer, whereas the d, e and fams represent modified aptamers incubated in human serum for different durations (2, 5 and 14 days). B74 (2-5 days), B76, B66, B71, B73 B03 (5-14 days) and, B79 (more than 14 days) were stable when compared to their respective buffer-treated controls.

FIG. 19: Evaluation of stability of WNV DIII modified aptamer B03 in fetal bovine serum (FBS). The stability of modified aptamer B03 reduces gradually with time for 4 days, beyond which no further reduction is observed. The same modified aptamer remains relatively stable for all 5 days of incubation.

FIG. 20: Binding of modified aptamer B03 to WNV DIII protein in the presence of human serum. After 24 hours of incubation, the aptamer still binds to the target protein.

FIG. 21: Binding of modified aptamer B03 to WNV in the presence of human serum or FBS. The results indicate that the modified aptamer is still functional after incubating with human and also FBS for up to 48 hours.

FIG. 22: Comparison of stability of WNV DIII side-chain modified and unmodified aptamers B03 in human serum (serum) and FBS. Side chain-modified aptamer B03 is highly stable whereas its unmodified DNA aptamer counterpart loses its stability after 24 hours of incubation in human serum and FBS.

FIG. 23: Comparison of stability of WNV DIII side-chain modified and unmodified aptamer B99 in human serum and FBS. Side-chain modified aptamer B99 is highly stable whereas its unmodified DNA counterpart loses its stability after 24 hours of incubation in human serum and FBS.

FIG. 24: Comparison of functionality of WNV DIII side-chain modified and unmodified aptamers. Side-chain modified aptamers B03 and B99 bind to WNV DIII protein whereas unmodified DNA aptamers B03 and B99 do not.

FIG. 25: Comparison of binding between different non-biotinylated modified aptamers and antibody, and WNV DIII protein. The modified aptamers were coated and their binding efficiencies evaluated using biotinylated WNV DIII protein (BNWNDIII).

FIG. 26: The cloning strategy for Dengue virus serotype 2 envelope protein domain III (DENV2-rEDIII). A. Schematic diagram showing the overlapping extension PCR (OE-PCR) technique used to obtain biotinylated DENV2-rEDIII (DENV2 BN-rEDIII) for downstream screening and evaluation of aptamers. Fragment 1 is designed such that its 3′ overhang is complementary to the 5′ overhang of Fragment 2. As such, primers B and C are complementary to each other. Both fragments are joined together via the complementary sequence and amplified by primers A and D. B. Construct of the recombinant DENV2-rEDIII protein generated using OE-PCR. The biotin acceptor peptide (BAP) is downstream of the 6×His tag and thrombin cleavage site, but upstream of the enterokinase cleavage site, whereas Fragment 2 encodes for the DENV2-rEDIII protein. 6×His tag at the N-terminal is used for affinity purification while BAP is the signal peptide for biotinylation. Thrombin and enterokinase cleavage sites are included to obtain the protein-of-interest without tags. C. Schematic representation of the construct showing the affinity tag, protease cleavage sites, biotinylation site and the protein-of-interest. A similar cloning strategy was used to obtain DENV1, 3 and 4 BN-rEDIII proteins.

FIG. 27: Schematic representation showing the step-by-step process involved in the production of DENV1-4 BN-rEDIII proteins for downstream screening and evaluation of aptamers.

FIG. 28: Production of purified biotinylated DENV2 BN-rEDIII protein. FPLC-SEC chromatography profile for purification of DENV2 BN-rEDIII protein. Both traces correspond to UV absorbance of protein at 280 nm (Broken line: DENV2 rEDIII, Continuous line: DENV2 BN-rEDIII). The traces do not overlap exactly due to molecular weight differences between the biotinylated and the non-biotinylated DENV 2 DIII protein. (B) Western blot analysis of DENV2 BN-rEDIII protein before (Lane 2) and after SEC purification (Lane 3: FPLC Purified Fraction 1; Lane 4: FPLC Purified Fraction 2). Asterisk (*) denotes the purified DENV2 BN-rEDIII monomeric protein).

FIG. 29: Peak-top heights of Biacore sensogram for different modified aptamers tested using surface plasmon resonance (SPR) against DENV2 rEDIII protein. Modified aptamers represented by the diamonds numbered 1-10 are chosen for further evaluation.

FIG. 30: Protein-coated ELMASA for modified aptamer affinity screening. The DENV2 rEDIII protein is coated on the maxisorp plate. There is significant binding by modified aptamers B006, B012 and B027 to DENV2 rEDIII protein as compared to the controls.

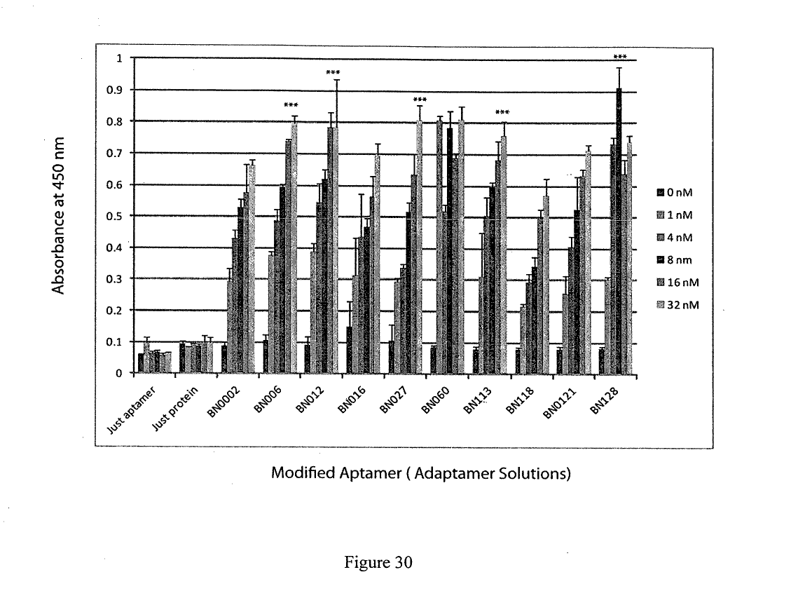

FIG. 31: Protein-coated ELMASA for modified aptamer affinity screening. The DENV 1 rEDIII protein is coated on the maxisorp plate. There is no significant binding by all 10 modified aptamers tested to DENV1 rEDIII protein as compared to the controls.

FIG. 32: Protein-coated ELMASA for modified aptamer affinity screening. The DENV3 rEDIII protein is coated on the maxisorp plate. There is no significant binding by all 10 modified aptamers tested to DENV1 rEDIII protein as compared to the controls.

FIG. 33: Protein-coated ELMASA for modified aptamer affinity screening. The DENV4 rEDIII protein is coated on the maxisorp plate. There is no significant binding by all 10 modified aptamers tested to DENV1 rEDIII protein as compared to the controls.

FIG. 34: DENV2 coated ELMASA for modified aptamer affinity screening. The wildtype DENV2 is coated on the maxisorp plate. Modified aptamers B118, B121 and B128 bind significantly at all the concentrations tested when compared with the controls. In contrast, the other modified aptamers only bind to the virus significantly at concentrations higher than 4 nM.

FIG. 35: Percentage neutralization of DENV2 by 1 μM of modified aptamers. Modified aptamers B60, B121 and B128 significantly block virus entry by binding to the envelope protein of DENV2.

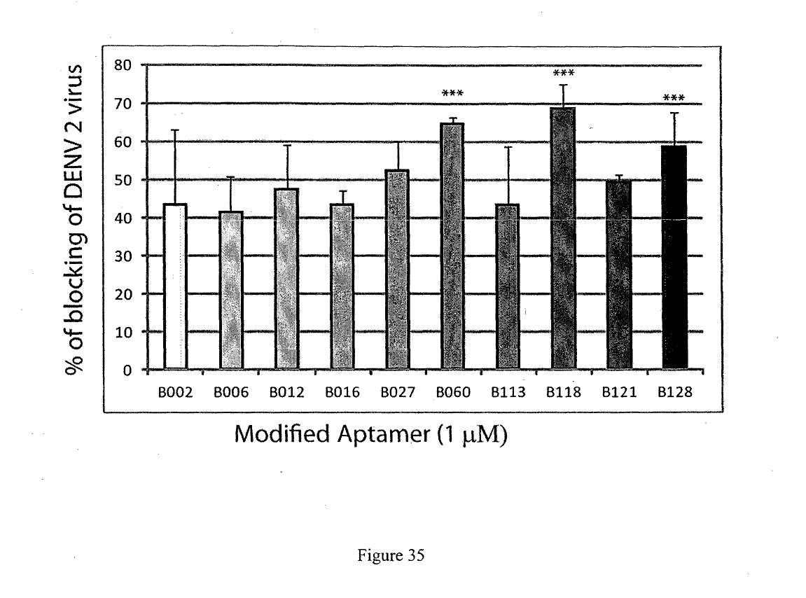

FIG. 36: Protein-coated ELMASA for determination of potential cross-reactivity against other flaviviruses. (A) WNV DIII, (B) TBEV-281 or (C) JEV-290 envelope protein is coated on the maxisorp plate. The modified aptamers do not cross-react with WNV DIII, TBEV and JEV envelope proteins.

FIG. 37: Protein-coated ELMASA for aptamer affinity screening. The rEDIII proteins of DENV1-4 and WNV, and the envelope proteins of TBEV (TBEV-281) and JEV (JEV-290) are coated on the maxisorp plate to test the binding of the commercial aptamer (D2A). Aptamer D2A does not confer any binding activity to all the flavivirus envelope or DIII proteins tested.

FIG. 38: Protein-coated ELMASA for evaluation of cross-reactivity of modified aptamer B128 with other flavivirus envelope or EDIII proteins. The DENV1-D4 rEDIII, WNDIII, or the envelope proteins of TBEV and JEV are coated on the maxisorp plate. Modified aptamer B128 only binds significantly to DENV2 rEDIII protein but not the rest of the target proteins tested.

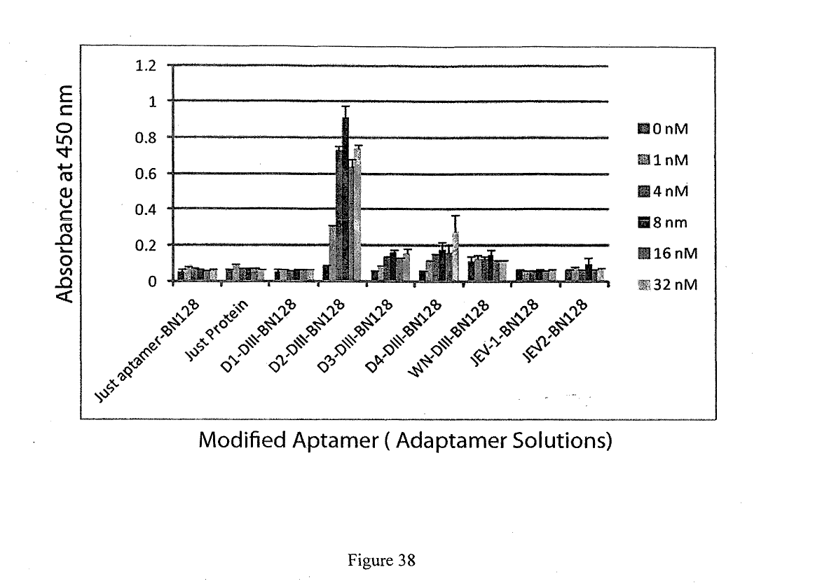

FIG. 39: Schematic representation showing the step-by-step process involved in the evaluation of modified aptamers against DENV2 rEDIII protein.

The present invention aims to develop a new platform using modified aptamers for diagnostic and therapeutic applications to flaviviruses, in particular West Nile and Dengue viruses.

Advantageously, the present invention utilizes a modified aptamer rather than the conventional DNA or RNA aptamer, whereby the DNA strands contain modified amino acid side-chains. These amino acid side chains form additional intermolecular interactions between the aptamer and target protein, thus resulting in higher affinity interactions. The modified aptamer technology may be used to develop new therapeutics, as well as a new platform for the diagnosis of flavivirus infections.

As a proof of concept, the West Nile virus and Dengue virus serotype 2 envelope Domain III (DIII) proteins were used as antigens/target proteins for the designing of modified aptamers. For each protein, binding of the protein was screened against a random library of 10¹³ aptamers, followed by identifying the specific and strong binding aptamers to each of the proteins. By evaluating the binding characteristics of the selected aptamers with each of the purified DIII protein and the full length E protein in the virus, aptamers that can be utilized for diagnostic and therapeutic applications were identified. Ten potential aptamer candidates for each protein were evaluated and the results are discussed below.

### DETAILED DESCRIPTION OF THE INVENTION

The invention will now be further described with reference to the following non-limiting examples.

### Example 1: Evaluation of West Nile Virus (WNV) Envelope DIII Protein Modified Aptamers

Material and Methods

Construction of pET28a WNE-BNDIII Plasmid

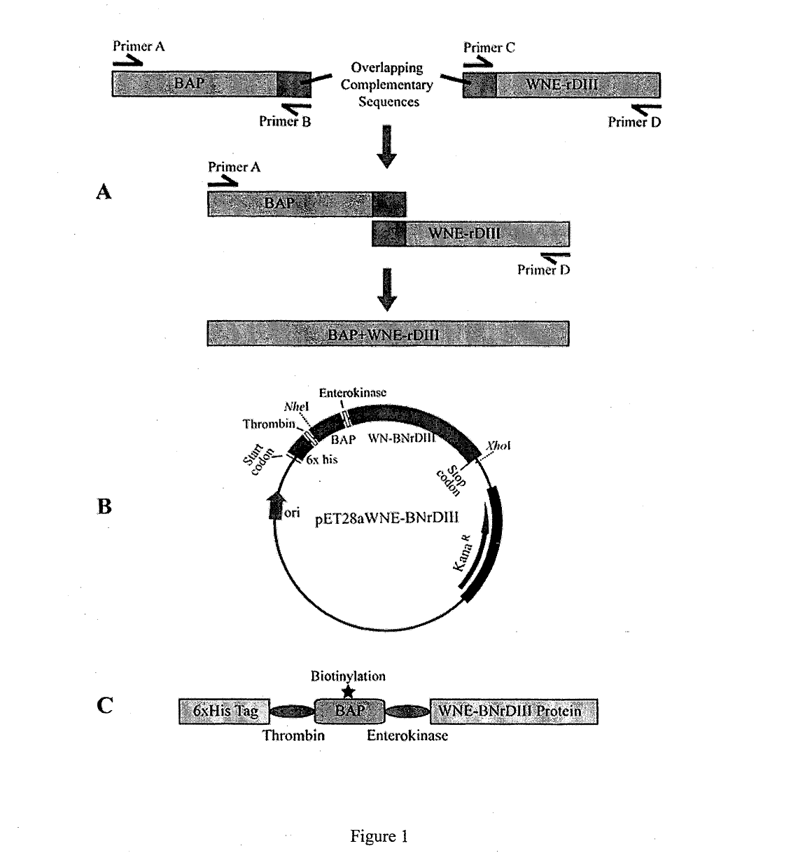

WNE-DIII gene (Wengler strain) was sub-cloned from the lab original plasmid which harbors the WN-DIII gene. The DIII gene was previously amplified from cDNA synthesized from West Nile virus Wengler strain. Primers Biotin_F, BiotinWNDIII_F, Biotin_WNDIII_R, and WNDIII_R (Table 1) were used to join the biotinylation signal peptide gene containing an enterokinase cleavage site with the WNEDIII gene via overlap extension PCR (OE-PCR) as shown in FIG. 1. Gel-purified PCR products containing the joined fragments were subsequently cloned into pET28a expression vector (Novagen, Germany) via NheI and XhoI cut sites. 6×His tag and thrombin cleavage site are at the N-terminus of the biotinylation signal peptide followed by enterokinase cleavage site and WNDIII protein at the C-terminus. DNA sequencing was performed to verify the constructs.

TABLE 1
List of primers used for cloning of biotinylated
WNV DIII proteins.

| Primers | Description | Sequence (5′-3′) |
|---|---|---|
| 1. Biotin_F | Forward primer | CTAGCTAGCTCCGGCC |
| | for priming out | TGAACGAC |
| | signal peptide | (SEQ ID No. 7) |
| | with NheI cut site | |
| 2. Biotin_ | Forward primer for | GACGACGACAAGAGCC |
| WDIII_F | overlapping signal | TGAAGGGAACATATGG |
| | peptide and WNV | (SEQ ID No. 8) |
| | DIII protein | |
| 3. Biotin_ | Reverse primer for | TGTTCCCTTCAGGCTC |
| WDIII_R | overlapping signal | TTGTCGTCGTC |
| | peptide and WNV DIII | (SEQ ID No. 9) |
| | protein | |
| 4. WDIII_R | Reverse primer for | CCGCTCGAGTTAGCTC |
| | priming out | CCAGATTTGTGCCA |
| | WNV DIII | (SEQ ID No. 10) |
| | protein from WNV | |
| | cDNA | |

Letters in BOLD are restriction enzyme recognition sites while underlined letters are overlapping PCR sites.

Protein Expression and Extraction.

pET28aBNWNDIII plasmid was transformed into BL-21-DE3 expression competent cells (Agilent Technologies, USA) and grown on Luria-Bertani (LB) agar containing 30 μg/ml kanamycin. Selected clones were cultured in 1 L LB broth (30 μg/ml kanamycin) at 30° C. until an absorbance OD₆₀₀ of 0.6. Expression of BN-WNDIII protein was induced with 1 mM isopropyl β-D-thiogalactoside (IPTG) overnight at 16° C. Bacterial cells were pelleted down with centrifugation at 8,000 rpm for 15 mM at 4° C. The protein expressed was targeted to inclusion bodies. In order to isolate the inclusion bodies, the pellet was resuspended in lysis buffer (20 mM Tris pH 8.0, 500 mM NaCl, 10 mM imidazole), followed by sonication in ice bath (15 mM, 10 Amp). The lysate was centrifuged at 12,000 rpm for 15 min at 4° C. A small white translucent pellet of inclusion body was obtained. The inclusion body pellet was then washed with the same lysis buffer followed by incubation with extraction buffer (8 M urea, 20 mM Tris, 300 mM NaCl, 10 mM imidazole, pH 8.0) at room temperature for 30 mM. The lysate was subsequently clarified by centrifugation at 13,500 rpm for 20 min.

Purification.

The extracted inclusion body containing the BN-WNDIII protein was incubated with nickel-nitrilotriacetic acid (Ni-NTA) resin (Bio-Rad, USA) for binding in a chromatography column overnight at 4° C. Ten column volumes of wash buffer (8 M urea, 20 mM Tris, 300 mM NaCl, 20 mM imidazole, pH 8.0) was used to wash away non-specific binding proteins. BN-WNDIII protein was eventually eluted out with elution buffer (8 M urea, 20 mM Tris, 300 mM NaCl, 500 mM Imidazole, pH 8.0) in six fractions. Next, all eluates were combined for refolding. Briefly, eluates were pooled into a SnakeSkin dialysis membrane tubing (Thermo Scientific, USA) and 0.5% of Tween-20 was added into the samples. The dialysis tubing was incubated in 1 L of 6 M urea for 6-12 hrs at 4° C., and 250 ml of 25 mM Tris (pH 8.0) was added into the solution every 6-12 hrs. When the final volume reached 3 L, the dialysis tubing was transferred into 2 L of 25 mM Tris and 150 mM NaCl (pH 8.0) for 6 hr. Refolded WNDIII protein was collected from the dialysis tubing. Fractions containing the protein-of-interest were injected into a FPLC machine and further purified via size-exclusion chromatography in PBS.

Protein Identity Analysis.

Samples collected from the flow through, wash, and eluates were analyzed by SDS-PAGE and Western blot. 12% Tris-tricine polyacrylamide denaturing gel was used to separate proteins in the samples and it was subsequently stained with Coomassie blue for protein detection. The presence of biotinylated WNDIII protein was confirmed by Western blot via two different approaches. First, the identity of WNDIII protein was determined with anti-His antibody. Briefly, separated proteins were transferred from the polyacrylamide gel onto a PVDF membrane using iBlot® Dry Blotting System (Life Technologies, USA). Blocking was done with 5% BSA for 1 hr at room temperature. Next, the membrane was incubated with 0.1 μg/ml mouse anti-His antibody (Qiagen, Germany) overnight at 4° C. The membrane was then washed with 1×TBST and incubated with 0.1 μg/ml goat anti-mouse secondary antibody conjugated with HRP (Thermo Scientific, USA) for 1 hr at room temperature. After washing with 1×TBST, the membrane was developed using SuperSignal® West Pico chemiluminescent substrate (Thermo Scientific, USA). For the second approach, WNDIII protein was detected directly using streptavidin conjugated with HRP. After transferring the samples onto a PVDF membrane, it was blocked with 4% BSA for 1 hr at room temperature. Then, the membrane was incubated with HRP-conjugated streptavidin (Millipore, USA) for another hour at room temperature. Subsequently, the membrane was washed thoroughly with 1×PBST for 1 hr at room temperature and developed with chemiluminescent substrate. A similar purification procedure was used for the production of non-biotinylated WNDIII.

Sample Preparation for Mass Spectrometry.

Purified protein (BN-WNDIII and WNDIII) was electrophoresed through SDS-PAGE using 12% Tris-tricine polyacrylamide denaturing gel and stained with Coomassie blue. The background of Coomassie-stained gel was removed with destaining solution (40% methanol, 10% glacial acetic acid, 50% distilled H₂O). The BN-WNDIII protein-corresponding band was excised from the gel and kept in eppendorf tube containing distilled water. Samples were submitted to Protein and Proteomics Centre, Department of Biological Sciences, NUS for mass spectrometry analysis.

Enzyme Linked Immunosorbent Assay (ELISA) for Biotinylation.

Samples and standards were added into the wells of a MaxiSorp plate (eBioscience, USA) in triplicate. The plate was covered with aluminum foil and incubated for 2 hrs. All incubating and washing steps were carried out at room temperature. After washing with 1×PBST, blocking buffer was added into each well and incubated for another hour. Next, streptavidin-HRP enzyme conjugates was added and incubated for 1 hr. The plate was washed with 1×PBST to remove unbound conjugates and then substrate solution, tetramethyl benzidine (TMB), was added for development. The reaction was stopped by adding 0.5 M H₂SO₄ solution. The absorbance was measured immediately at 450 nm. Every batch of FPLC purified BN-WNDIII protein was tested by ELISA to ensure that the protein is biotinylated.

Biotinylated Protein Binding Assay.

The binding affinity of purified biotinylated WNDIII protein was tested using streptavidin magnetic beads (GE Healthcare, UK) according to manufacturer's protocol. Briefly, samples were mixed with streptavidin magnetic beads and incubated for 30 min with gentle mixing. Unbound proteins were removed with wash buffer while biotinylated proteins were eluted out with elution buffer provided in the kit. Eluted proteins were analyzed by Western blot and ELISA.

Selex procedure for aptamer designing: Apta Biosciences Pte Ltd, (Adaptamer Biosolutions) www.aptabiosciences.com, 31 Biopolis Way, #02-25 Nanos, Singapore 138669, Phone: +65-3109-0178, Fax: +65-6779-6584, Mobile+65-9184-7323) formerly known as Fujitsu Biolaboratories. Bio-laboratories, R&D Division, (Fujitsu Asia Pte Ltd, Fujitsu Laboratories Ltd., Nanotechnology Business Creation Initiative, 31 Biopolis Way, #02-25 Nanos, Singapore).

Aptamer designing and synthesis: Fujitsu, Biolaboratories, Singapore.

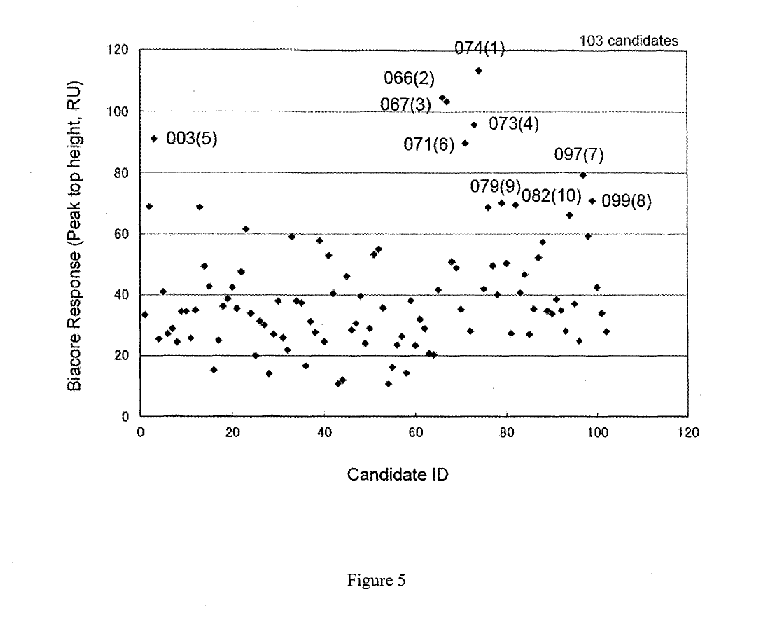

Surface Plasma Resonance (SPR) Analysis using BN-WNDIII protein: Fujitsu (FIG. 5)

SPR analysis using WNDIII for affinity calculation: Fujitsu (Table 2)

Ten aptamers received from Fujitsu for evaluation are as follows:

Non-Biotinylated aptamers: N03, N66, N67, N71, N73, N74, N76, N79, N97, N99

Biotinylated aptamers: B03, B66, B67, B71, B73, B74, B76, B79, B97, B99

TABLE 2
List of aptamers chosen for further evaluation after
measurement of their affinities using SPR.

| | KD at pH | KD at pH |
|---|---|---|
| Aptamer ID | 5.5 (nM) | 5.0 (nM) |
| WNDIII-003 | 8.5 | 11.4 |
| WNDIII-066 | 15.2 | 12.6 |
| WNDIII-067 | 16.0 | 9.6 |
| WNDIII-071 | 32.0 | 9.9 |
| WNDIII-073 | 23.8 | 12.7 |
| WNDIII-074 | 25.6 | 10.9 |
| WNDIII-076 | 25.0 | 13.9 |
| WNDIII-079 | 23.4 | 14.2 |
| WNDIII-097 | 30.9 | 10.7 |
| WNDIII-099 | 25.8 | 8.8 |

Enzyme Linked Modified Aptamer Sorbent Assay (ELMASA) for Surface Screening.

The modified aptamers consist of amino acid side-chains incorporated into the DNA backbone in order to enhance the binding of the aptamer molecule to the target protein. In order to select the suitable surface for the analysis of the modified aptamer, four different ELISA surfaces were tested. (Nunc-Multisorp, Polysorp, Medisorp and Maxisorp). Briefly, 50 ng of biotinylated aptamer and different concentrations of BN-WNDIII proteins were added to each well and incubated at 4° C. overnight. Blocking with 4% BSA was carried out after overnight incubation, followed by washing with PBS. Then, 1:2000 dilution of streptavidin-HRP enzyme conjugates was added and incubated for 1 hr. The plate was washed 6 times with 1×PBST to remove unbound conjugates. Then, tetramethyl benzidine (TMB) substrate solution was added for development and incubated for 15 min at room temperature. 0.5 M H₂SO₄ solution was added to stop the reaction. The absorbance was measured immediately at 450 nm.

Protein Coated Enzyme Linked Modified Aptamer Sorbent Assay for Affinity Screening.

100 ng of purified non-biotinylated WNDIII protein was coated on maxisorp plate overnight at 4° C. Following the coating, the ELISA plate was washed three times with PBS and incubated for 1 hour with different concentrations (1.65 nM to 26 nM/well) of biotinylated aptamers solubilized in RNase free TE buffer (Invitrogen). Then, 1:2000 dilution of streptavidin-HRP enzyme conjugates was added and incubated for 1 hr following the standard procedure as mentioned above.

Virus Coated Enzyme Linked Modified Aptamer Sorbent Assay.

Instead of using DIII protein, West Nile virus Wengler strain was coated onto the ELISA plate. Briefly, 1000 PFU of virus was coated in each well followed by overnight incubation at 4° C. The wells were washed with 1× PBST followed by blocking with 4% BSA. Following this step, the wells were incubated with different concentrations (0.3 nM to 26 nM/well) of biotinylated aptamers (1-10) for 1 hr. Then, 1:2000 dilution of streptavidin-HRP enzyme conjugates was added and incubated for 1 hr following the standard procedure as mentioned earlier. Coating, Washing, aptamer addition and developing were carried out in the BSC class 2.

Plaque Reduction Neutralization Test (PRNT).

Baby hamster kidney (BHK) cells were seeded in a 24-well plate overnight before use. Frozen virus stocks were carefully thawed and diluted to 1000 PFU/ml. To 50 PFU/50 μl West Nile virus Wengler strain, various concentrations (1.25 nM, 2.5 nM, 5 nM, 10 nM, 20 nM, 40 nM, 80 nM, 165 nM, 330 nM, 660 nM, 13.33 μM, 5 μM and 10 μM/well) of non biotinylated aptamers were added in duplicates and allowed to incubate for 1.5 hrs for binding. Cell growth medium was removed from the 24-well plate, the cell monolayers briefly washed with 2% RPMI and then infected with 100 μl of the aptamer+virus incubated mixture. The plate was incubated at 37° C. and 5% CO₂ for 1 hr with constant rocking of the plate at every 15 min interval. The inoculum was aspirated, briefly washed with 2% RPMI and each well overlaid with 1 ml overlay medium. The plate was incubated at 37° C. and 5% CO₂ for 4.5 days until plaques were formed. The cell monolayer was stained with a solution of 0.1% crystal violet in PBS for 24 hrs. The crystal violet solution was removed, the plates washed in distilled water and plaques were counted.

Aptamer Stability Assay:

Stability of the aptamers was tested by incubating a fixed concentration (400 ng/ml) of aptamer at three different temperatures (−20° C., room temperature and 37° C.) for 1 to 5 days. After each time point, the integrity of the aptamers was analysed by running a 1.5% agarose gel which was premixed with GEL-RED. The sample was ran 40 V for 4 hr and viewed on a Gel-doc under ultraviolet (UV) light.

ApoTox-Glo Triple Assay.

The assay was performed using ApoTox-Glo Triple Assay kit (Promega) and readings were taken using Glomax Instrument. Briefly, BHK cells were seeded in a 96-well assay plate with cell density of 5000 cells/well (5000 cells/0.1 ml) and cultured overnight. After 24 hrs, cells were treated with aptamers (3.3 to 26 nM concentration/well) and positive controls for cytotoxicity (digitonin detergent, MPER, membrane protein extraction reagent). At day 1 and day 4, the cells were incubated with 20 μl of Viability/Cytotoxicity Reagent. The plate was briefly mixed by orbital shaking at 300 rpm for 30 seconds and incubated at 37° C. for 30 min. Fluorescence was measured at two wavelength sets, 400_{Ex}/505_{Em} (Viability) and 485_{Ex}/520_{Em} (Cytotoxicity). For luminescence reading, the plate was inoculated with 100 μl of Capase-Glo 3/7 Reagent in each well. The plate was briefly mixed by orbital shaking at 300 rpm for 30 sec and incubated at room temperature for 30 min.

Alamar Blue Viability Assay.

The assay was performed using alamarBlue Cell Viability Assay (Invitrogen) and readings were taken using Glomax Instrument. BHK cells were seeded in a 96-well assay plate with cell density of 5000 cells/well (5000 cells/0.1 ml) and cultured overnight. After 24 hrs, cells were treated with aptamers (3.3 to 26 nM concentration), and positive controls for cytotoxicity (digitonin detergent, MPER, membrane protein extraction reagent). At day 1, 2, 3 and 4, the cells were incubated with 10 μl of alamar Blue reagent. The plate was briefly mixed by orbital shaking at 300 rpm for 30 sec and incubated at 37° C. for 1-4 hrs, protected from direct light. Fluorescence of the plate was measured at 570_{Ex}/585_{Em}.

Determination of Stability of the Modified Aptamers in Serum by ELISA Method:

Known amount (40 ng/well) of biotinylated aptamers were coated on the Maxisorp plate and incubated at RT for 2 hours. Then the plates were incubated with and without 100% and 20% serum for varying time points (1, 20, 48 and 120 hours). Positive controls (Just aptamer) were incubated with 4% Bovine serum albumin (BSA). At the end of each time point the serum and BSA were removed. Streptavidin-HRP enzyme conjugates (1:5000 dilution) was added and incubated for 1 hr. The plate was washed 6 times with 1×PBST to remove unbound conjugates. Then, tetramethyl benzidine (TMB) substrate solution was added for development and incubated for 15 min at room temperature. 0.5 M H₂SO₄ solution was added to stop the reaction. The absorbance was measured immediately at 450 nm. As a negative control the aptamer (303) was boiled at 95° C. for 48 hours. If the aptamer is degraded by the serum or heating, then the aptamer will not be detected by the streptavidin-HRP.

### Results

### Construction of WNE-BNrDIII Plasmid

To obtain the biotinylated protein of West Nile virus envelope protein domain III (WNE-BNrDIII) for aptamer screening, a new plasmid construct was designed by engineering in the biotinylation acceptor peptide (BAP) on the N-terminus, and an enterokinase cleavage site between the BAP and the WNDIII gene. The DNA sequence corresponding to the BAP was chemically synthesized (Cull et al., 2000), whereas the WNDIII sequence was obtained from the previous construct, which was derived from the cDNA of WNV Wengler strain. Later, the BAP sequence with the enterokinase cleavage site was linked to WNDIII at the 5′ end through overlapping extension PCR (OE-PCR) as illustrated in FIG. 1A. The final PCR product and pET28a vector were double-digested with NheI and XhoI restriction enzymes and the recombinant gene ligated into the digested plasmid, which consists of a 6×His tag upstream of the multiple cloning site. Thus, the recombinant BAP-containing WNDIII envelope protein has been cloned with 2 tags at the N-terminus, namely the 6×His tag (for affinity purification) and biotin (to bind to streptavidin) and contains two enzyme (thrombin and enterokinase) cleavage sites (FIGS. 1B & 1C). This engineered construct was then transformed into E. coli TOP 10 and the positive clones were verified by colony PCR, restriction digestion and DNA sequencing. The novelty of the plasmid is that the biotin acceptor peptide (BAP) has been engineered with the WNDIII gene for biotinylation. This construct can be utilized for both in vivo and in vitro biotinylation. In addition, the thrombin and enterokinase cleavage sites enable removal of either or both tags after the purification. This allows the purified recombinant DIII protein to be used in downstream selection of protein interacting partners and/or aptamers from a pool of protein and/or aptamer library.

### Expression of WNE-BNrDIII Plasmid

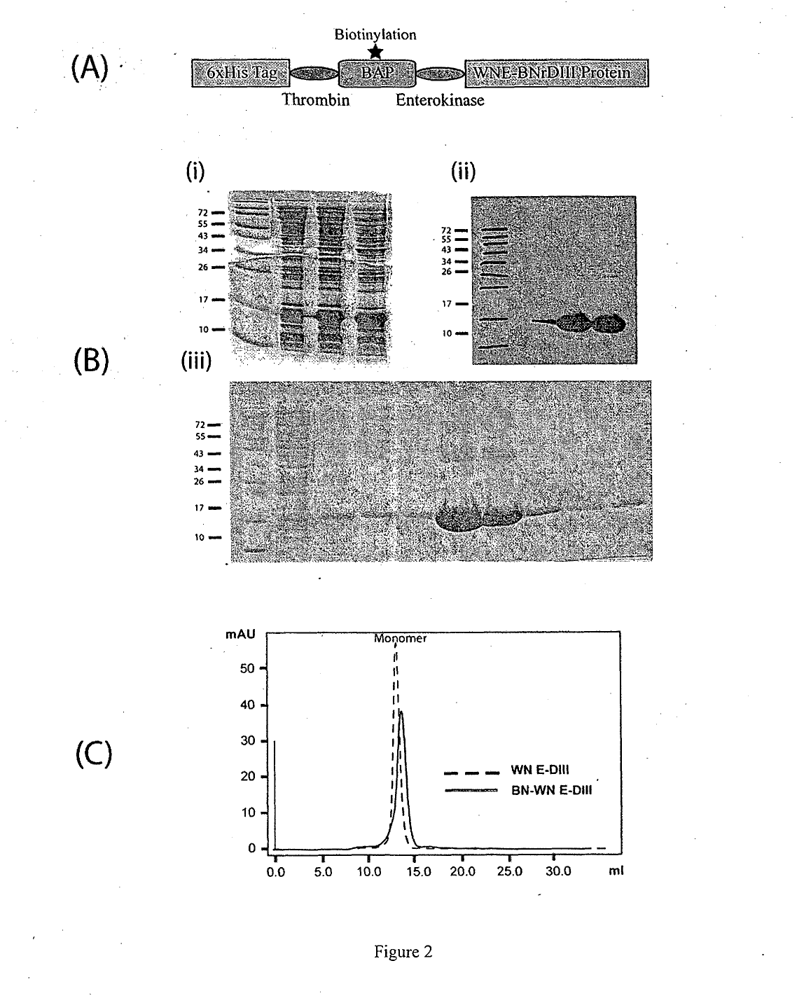

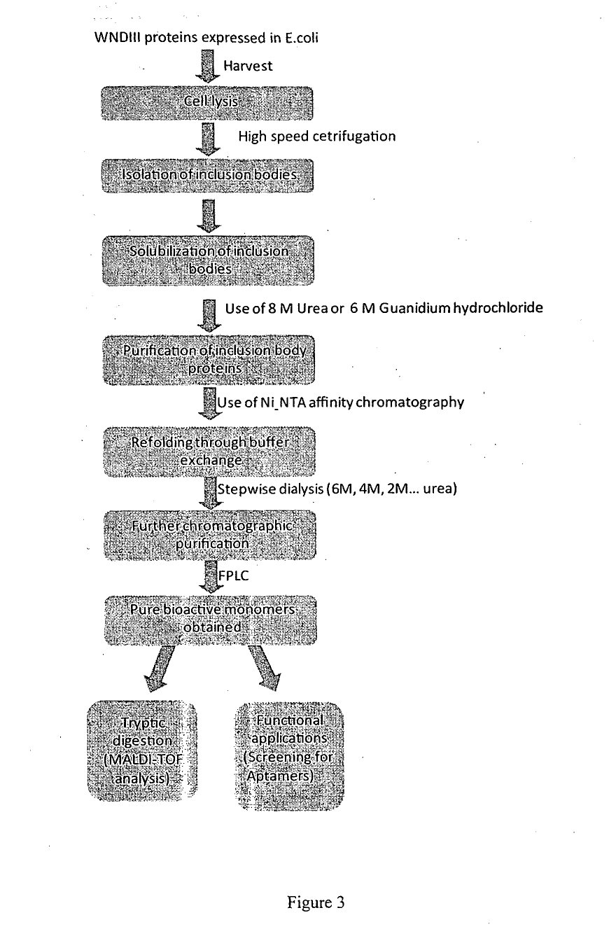

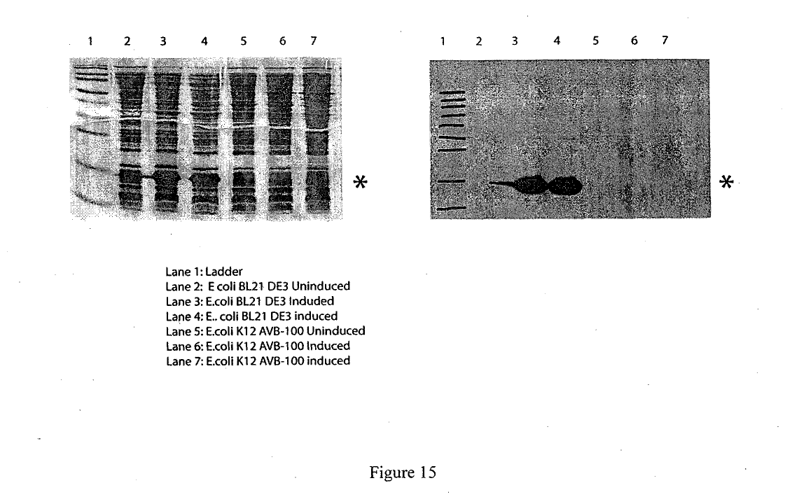

To express the recombinant protein, the engineered plasmid was transformed into a commercial E. coli strain AVB-100 obtained from Genecopoeia. The AVB 100 E. coli strain has been incorporated with an overexpressing BR A (Biotin ligase) gene within the genomic DNA. This enzyme specifically adds a biotin molecule to the lysine residue of the BAP. Initially, the protein (BAP-WNDIII) of interest was not expressed in E. coli K12 AVB-100 (FIG. 15). The reason could be due to the intrinsic property of the protein being expressed in other bacterial systems. Previously, the WNE DIII protein in BL-21 (DE3) was expressed. In order to overcome this problem, the strategy was altered to express the BAP-WNDIII construct in E. coli BL21 DE3 followed by in vitro biotinylation using BirA enzyme. When the construct was expressed in E. coli BL21 (DE3), an obvious band corresponding to the recombinant full-length BAP-WN rDIII protein was detected in the lysate of IPTG-induced BL-21 (DE) strain [FIG. 2(A)(i)]. Western blot probed using an anti-His antibody revealed that the recombinant protein-of-interest was expressed in E. coli BL-21 (DE) [FIG. 2(A)(ii)]. After expression was confirmed, the culture volume was scaled up for production of large amounts of recombinant protein. After culturing, the cells were induced with IPTG. The cells were harvested and the inclusion bodies (IB) isolated. The crude protein was then extracted from the IB and subjected to His-tag affinity purification, refolding, and size exclusion chromatography as explained in the Materials and Methods. The SDS-PAGE and FPLC profiles corresponding to the BAP-WNDIII protein are shown in FIG. 2 (A)(iii) and (B). For comparison, the trace corresponding to unbiotinylated WNDIII is shown. Using this purification procedure, 1 mg of purified protein was obtained from 1 L of culture. The identity of the purified protein was further confirmed by mass spectrometry by carrying out in-gel tryptic digestion followed by peptide mass fingerprinting. A schematic flowchart representing the expression, purification and evaluation of the recombinant protein is shown in FIG. 3.

### Screening of Biotinylated Proteins:

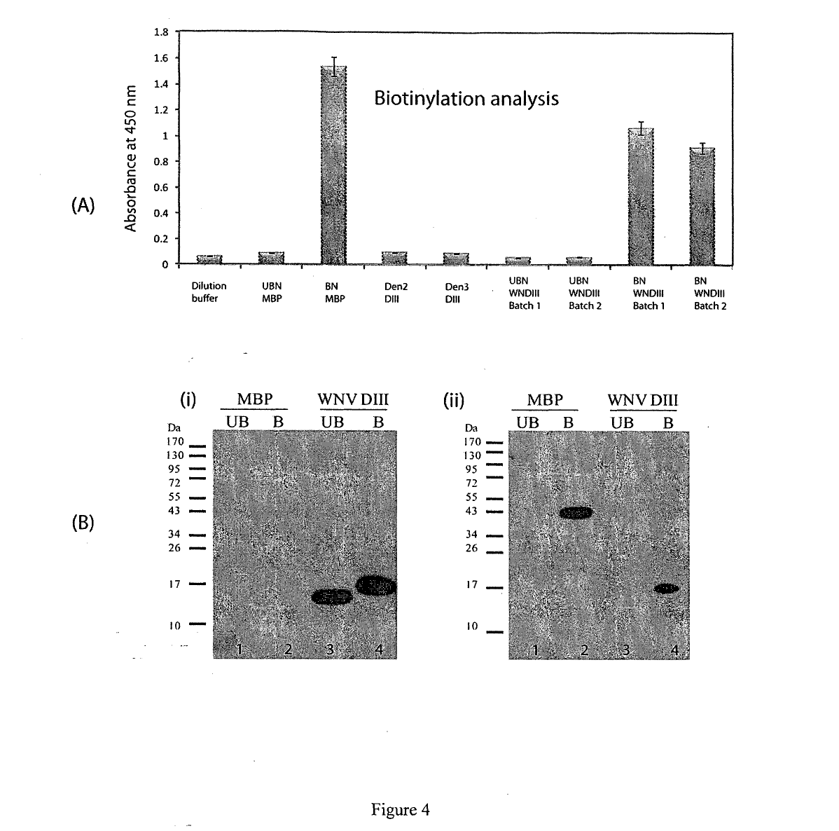

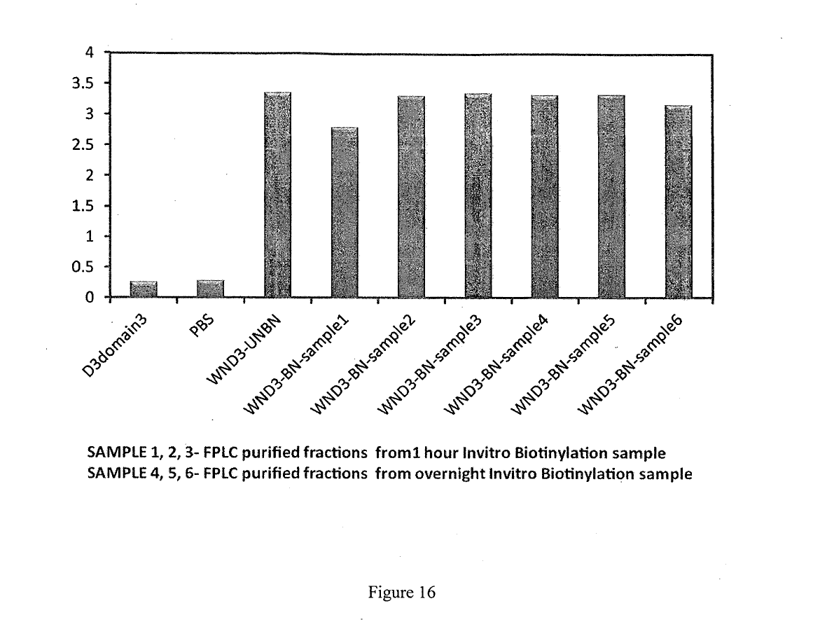

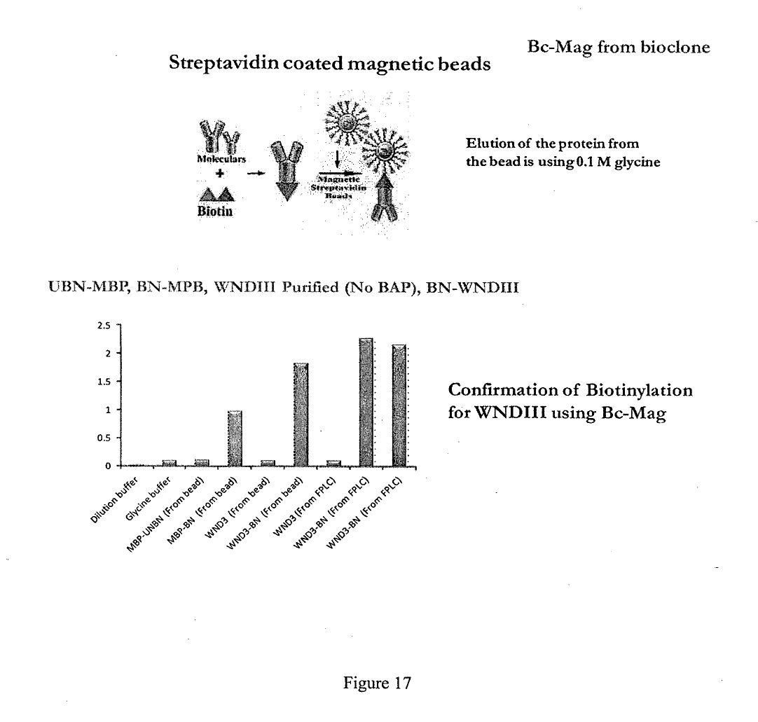

As the attempt to express the construct in K12 Strain AVB100 was unsuccessful, in vitro biotinylation using Bir-A enzyme was carried out. In vitro biotinylated WNDIII was tested using ELISA, and the result shows absorbance at 450 nm, indicating that the recombinant protein was biotinylated and binds to streptavidin-HRP conjugate in both experimental conditions (1 hr and overnight reaction set up). Interestingly, the control experiment, i.e. the sample without Bir A enzyme, also showed high absorbance at 450 nm, indicating that it also binds to the streptavidin-HRP conjugate (FIG. 16). To confirm that the endogenously in-vivo biotinylated WNDIII might be an artifact, the experiment was repeated thrice in ELISA. The positive and negative controls were used, i.e. biotinylated and unbiotinylated maltose binding protein (MBP), and WN-DIII and dengue 1-4 DIII proteins without BAP [FIG. 4(A)]. In all the tested conditions, the results obtained were the same, indicating that the BAP-WNDIII protein might be endogenously biotinylated. To further confirm this, tests via Western blot [FIG. 4(B)] using streptavidin-HRP conjugate and Bc Mag-streptavidin beads were carried out, and it was found that the protein was indeed endogenously biotinylated at the specific BAP site during expression (FIG. 17).

### Endogenous Biotinylation:

After it has been proved that the BAP containing WNDIII protein was endogenously in vivo biotinylated during the expression of the protein itself, there was an interest to understand how the biotinylation could have taken place endogenously, and where is the source for the biotin in the cell for the biotinylation. A bioinformatics search for the Bir A enzyme in the genomic DNA sequence of E. coli BL 21(DE3) was carried out and it was discovered that the gene encoding Bir A was found in the E. coli strain, which have been used for expression. In addition, biotin has been found to be present in the medium, which has been used to cultivate the bacterial cells (Tolaymat et al., 1989). Thus, the protein is endogenously biotinylated by the Biotin ligase enzyme already present in the cell, utilizing the biotin in the culture medium. Therefore, attaching a BAP to a gene-of-interest and expressing it in E. coli BL 21 (DE3) will result in the production of biotinylated protein endogenously, hence eliminating the need for a commercial expression strain or in vitro biotinylation. Thus, a platform to obtain endogenously biotinylated, purified protein for biological applications, like aptamer screening, has been established. Every batch of purified protein for biotinylation was checked and was found to be consistent. It was also tested to determine whether endogenous biotinylation is universal for other proteins by cloning the BAP for dengue virus capsid protein and it was confirmed that the capsid protein was found to be endogenously biotinylated. This showed that this platform can be potentially extended to other biotinylated proteins, which have commercial applications in diagnostics and drug development. This has been filed as a provisional patent by Exploit Technologies (Singapore Patent Application No. 201208602-1, Entitled: Biotinylated Protein, Filing Date: 22 Nov. 2012, contents of which are incorporated herein by reference).

### Evaluation of Modified Aptamers

### Surface Selection.

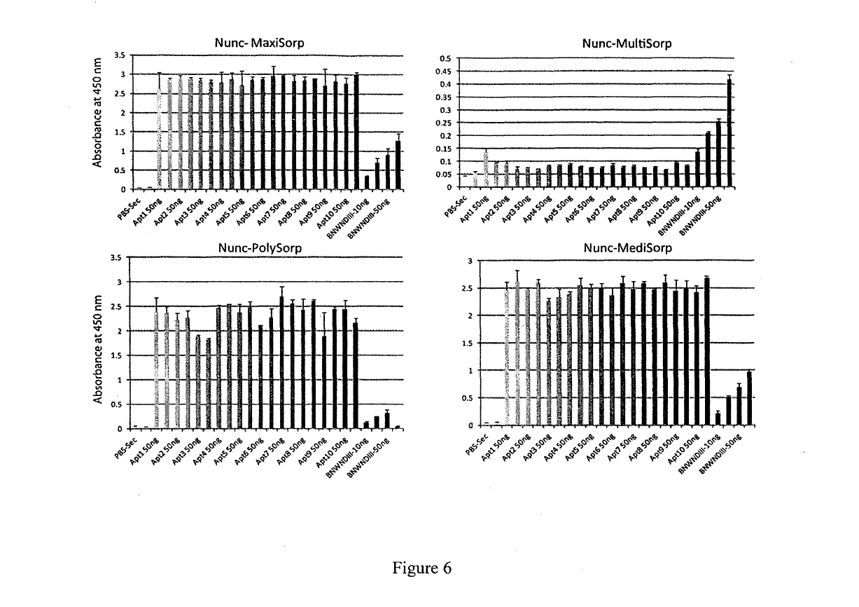

In order to test the binding efficiency of aptamers, suitability of the four different surfaces were tested by coating with 50 ng biotinylated modified aptamers (1 to 10) followed by detection with streptavidin-HRP conjugate. Similarly, varying concentrations of biotinylated WNDIII (10, 25, 50 and 100 ng/well) protein was also coated. The results are shown in the FIG. 6. In spite of the fact that all the experimental conditions were the same for the four different surfaces, differences in binding were observed. In the Multisorp plate, the absorbance at 450 nm indicated very low binding of the aptamers and WNDIII protein (maximum absorbance at 0.15 for aptamer and 0.1 to 0.5 for protein). The binding efficiencies for the Polysorp and Medisorp plates were found to be similar for aptamers (maximum absorbance varied from 2 to 2.5) whereas for WNDIII protein, it ranges from (0.1 to 1). For the Maxisorp plate, the absorbance for aptamers varied from 2.5 to 3 and from 0.2 to 1.3 for the protein. Thus, Maxisorp plate was selected as a good surface for coating aptamers as well as proteins for the further evaluation.

### Protein-Coated Enzyme Linked Modified Aptamer Sorbent Assay for Affinity Screening.

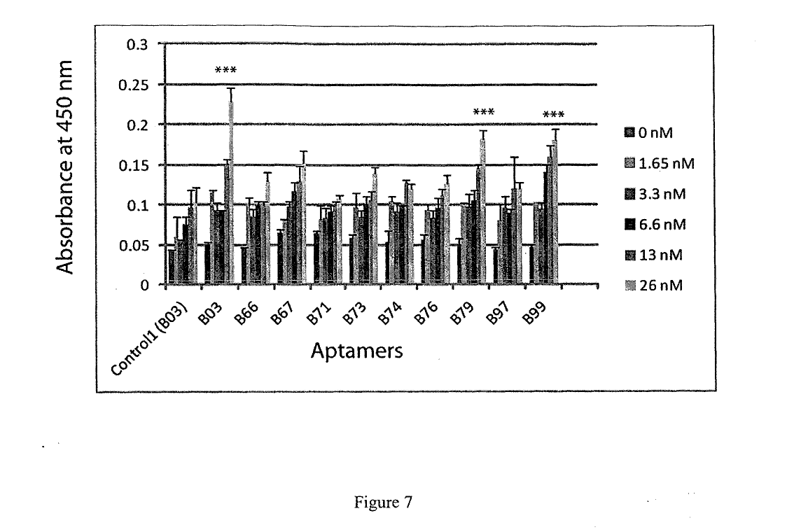

In order to evaluate specific binding of aptamers to WNDIII protein, protein-coated ELISA was carried out for the ten aptamers. WNDIII protein (100 ng/well) was coated overnight and incubated with biotinylated aptamers of various concentrations (0 to 26 nM), followed by probing with streptavidin-HRP conjugate. If an aptamer were to bind to the WNDIII protein, it would be detected through the enzyme substrate reaction. In this case, it was observed that aptamers B03, B79 and B99 bound to the WNDIII protein as their absorbance were significantly higher when compared to the control and the other aptamers (FIG. 7, indicated by asterisk). When various concentrations of aptamers were compared, the aptamers B03, B79 and B99 bound significantly (P<0.05) in all concentrations (3.3, 6.6, 13, and 26 nM) except 1.65 nM concentration. This indicated binding might be insignificant at 1.65 nM. The other aptamers bound less significantly to the WNDIII protein at various concentrations tested where absorbance was comparatively lower (0.05<p-value<0.1) when compared to B03, B97 and B99 as shown in the FIG. 7.

### Virus-Coated Enzyme Linked Modified Aptamer Sorbent Assay.

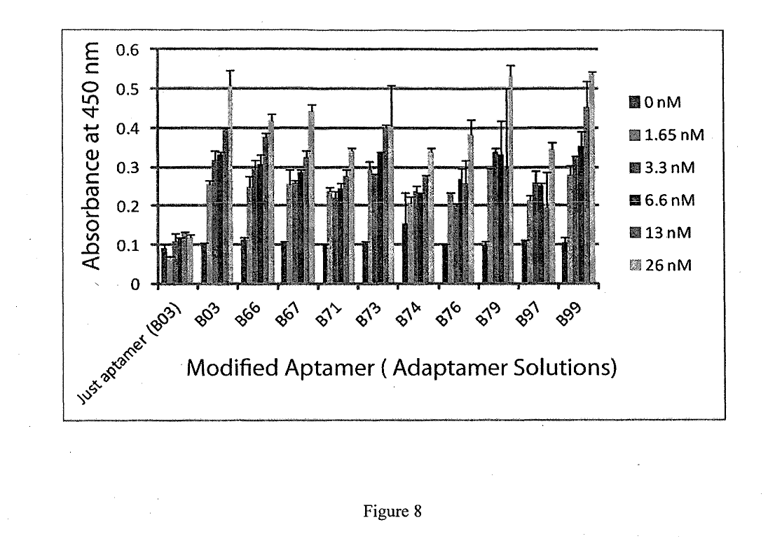

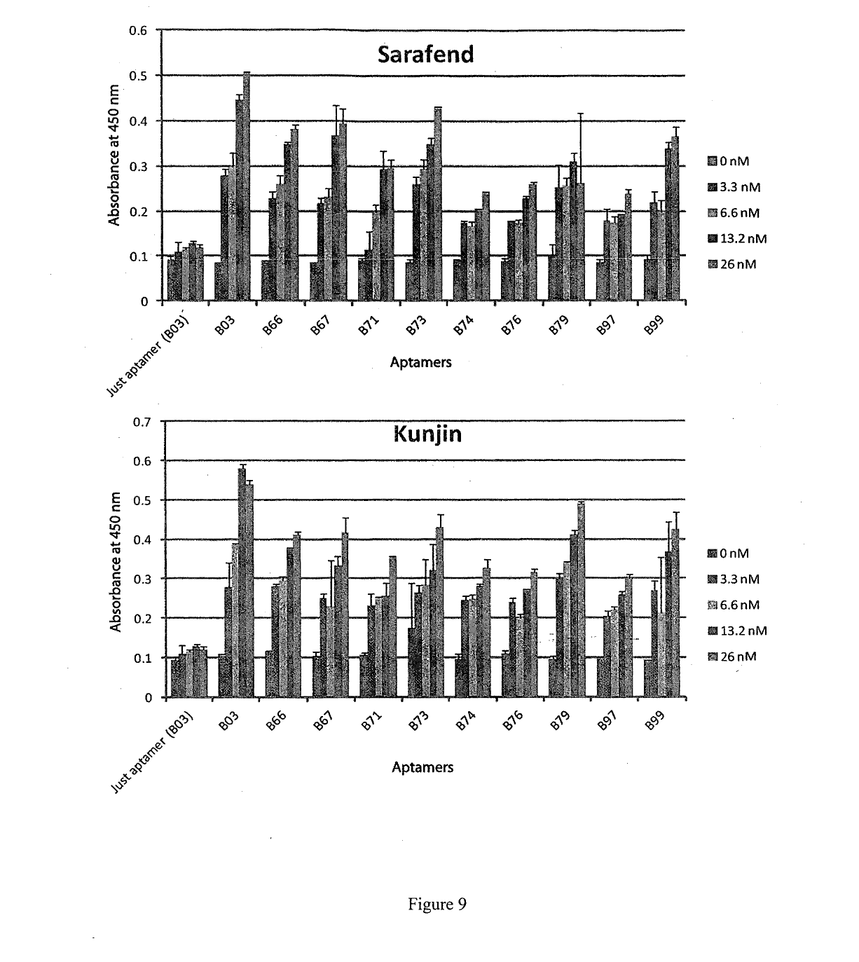

Once it had been confirmed that a modified aptamer was able to bind to purified WNDIII protein, it was evaluated whether the aptamer could bind to the West Nile envelope protein if the whole virus was coated. West Nile virus Wengler strain (1000 PFU/well) was coated in the ELISA plate overnight, followed by incubating with different concentrations of aptamers. It was still observed that the aptamers B03, B79 and B99 bind specifically to domain III in the native envelope protein present on the virus (FIG. 8). When various concentrations of aptamers were compared, aptamers B03, B79 and B99 bound significantly (P<0.05) for all the concentrations (0.3 nM to 26 nM) when compared with the control. In the case of other aptamers, it was found that they bind to the virus significantly in concentrations higher than 3.3 nM. This proved that the B03, B79 and B99 have higher binding efficiencies even at low concentrations when compared to the other aptamers. The intensities of the absorbance were generally higher in the case of virus-coated enzyme linked modified aptamer sorbant assay when compared to its protein-coated counterpart. This could be due to the availability of more envelope proteins in the virus for the aptamers to bind, ultimately leading to a higher absorbance. Negative control BSA and buffer controls were used and found that their absorbance were negligible. This result showed that these modified aptamers can bind specifically to the native domain III on wildtype West Nile virus. These modified aptamers can also bind to other West Nile virus strains namely, Sarafend and Kunjin virus strain (FIG. 9). Aptamers B03, B67, B73 and B99 bound significantly at concentrations higher than 3.3 nM to the Sarafend strain, while aptamers B03, B66, B67, B73 and B79 bind significantly at the concentrations higher than 3.3 nM to the Kunjin strain. These results indicated that the modified aptamers developed can be used for detection of different strains of West Nile viruses. A prototype aptamer based diagnostic can be built using two different aptamers. One unlabeled aptamer is attached to a surface to which a test sample can be added. Thus the first aptamer will bind to the antigen, which can then be detected using a second biotinylated aptamer (for ELISA or cassette for detection) or fluorophore attached to the aptamer (by imaging, microfluidics, or micro capillary detection). As such, application of aptamers can be expanded for diagnostic purposes for flaviviruses and also for identifying their different strains.

### Neutralization of West Nile Virus by Modified Aptamers.

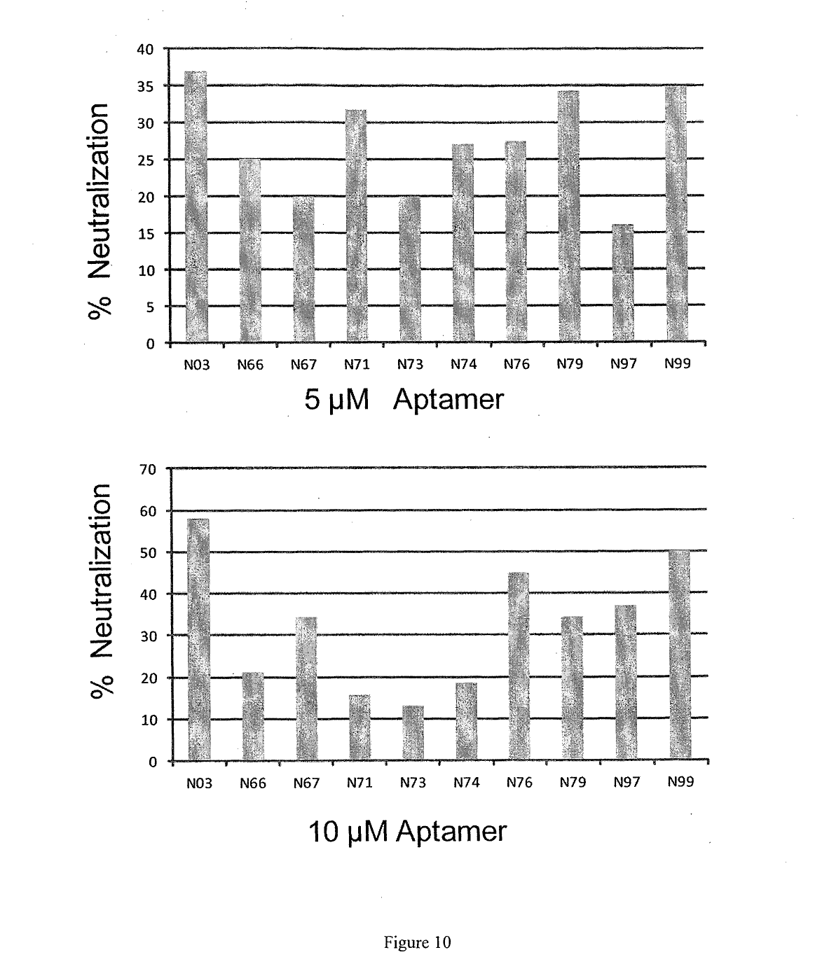

As it has been established that the modified aptamers were able to bind to purified WNDIII and native DIII in the envelope protein of wildtype West Nile virus, the ability of the aptamers to neutralize WNV was then tested. The virus was incubated with different concentrations of aptamers followed by infecting BHK cells with the aptamer-treated or untreated virus. Both the treated and untreated virus were removed after an hour. The plate was stained on day 4 after the infection and formation of plaques were observed. In the lower concentrations of aptamer treatment, there was no neutralizing activity. There was visible reduction in the number of plaques in the 5 μM and 10 μM aptamer treatment. FIG. 10 shows the percentage of neutralization obtained for the different tested concentrations. It was observed that 5 μM treatment of N03, N71, N79 and N99 showed about 30-35% neutralization whereas the other aptamers showed less than 30% neutralization. When the aptamer treatment concentration was 10 μM, N03 and N99 showed neutralization higher than 50%. These results showed that N03 and N99 have the potential to be developed as a therapeutics against West Nile virus.

### Viability Assay for Modified Aptamers

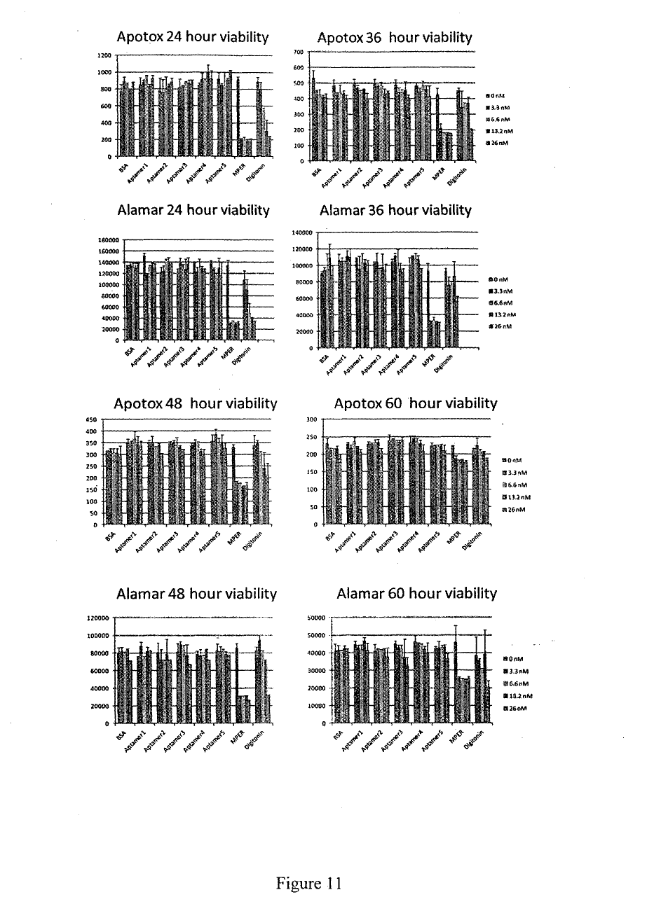

As the possibility for aptamers to be developed for therapeutics is very high, it was tested whether treating mammalian cells with the modified aptamers causes cytotoxicity to the cells. In order to check the outcome of cell viability during aptamer treatment, two different sets of viability experiments were performed. The first involved the use of the apotox-glo triple assay while the second involved the use of the alamar blue viability assay. The cells were treated with different concentrations of aptamers followed by testing the viability at various time points (24, 36, 48 and 60 hours post-treatment). The results obtained by the two methods are shown in FIG. 11. The results showed that, at the tested concentrations, the aptamers did not show any cytotoxicity and the cells were still viable compared to that of normal untreated cells. The positive controls like MPER and digitonin treatment showed that cell viability was lost. The result was comparable to that of the viability assay carried out using alamar blue. Thus, the combined results indicated that under the tested conditions of 3.3 to 26 nM, the cells were viable like the untreated cells, up to 60 hrs post-treatment.

### Aptamer Stability Assay:

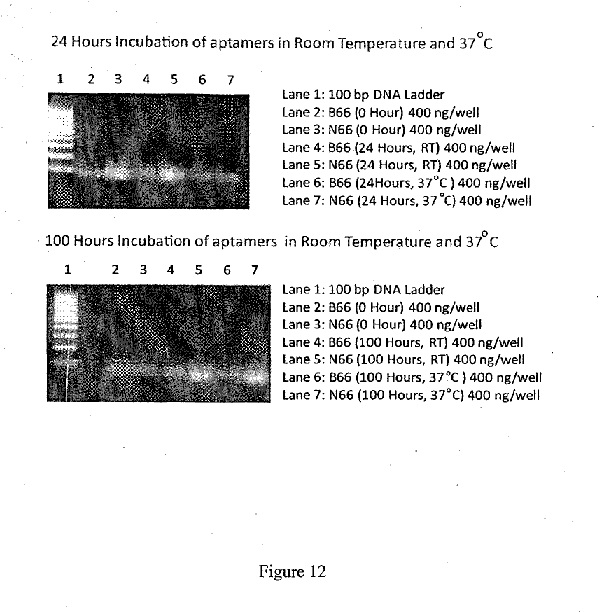

The stability of the aptamers were tested by incubating them at three different temperatures (−20° C., room temperature and 37° C.) for different periods of time (1 to 5 days), followed by checking the integrity of the modified aptamers in a gel-red stained agarose gel. FIG. 12 shows that the aptamers which were incubated for 5 days at room temperature and 37° C. were still stable and intact and their corresponding bands could be detected by gel red.

### Aptamer Stability in Human Serum:

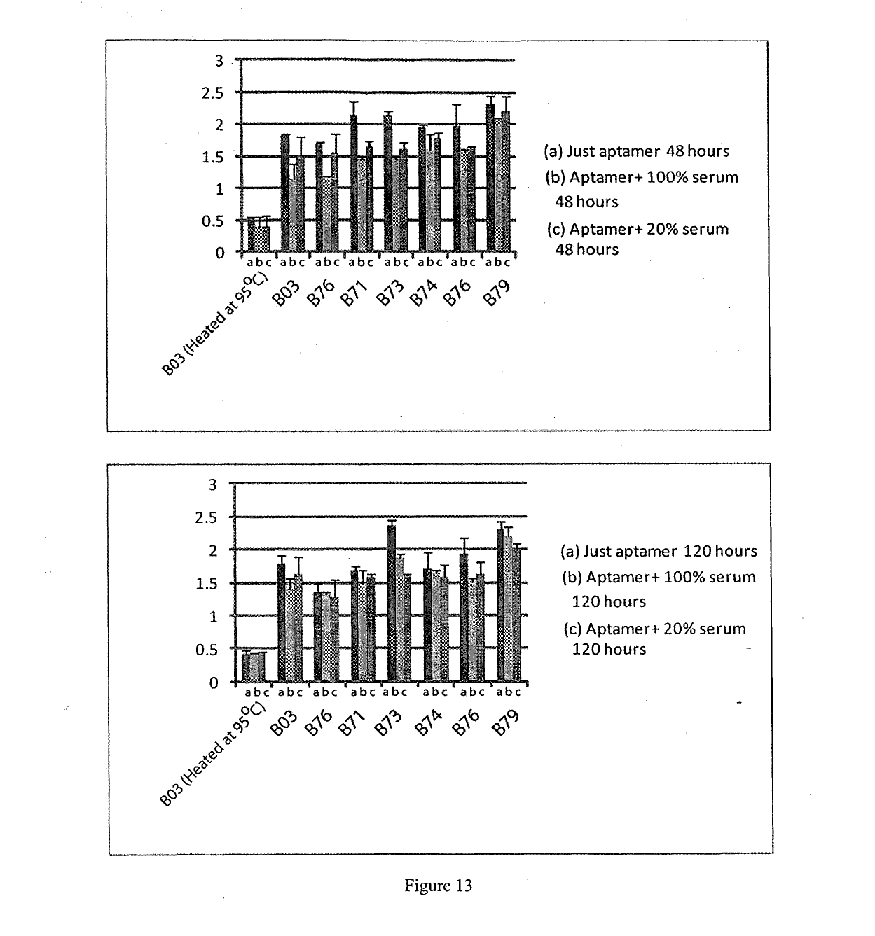

Testing the stability of the modified aptamers was extended in the presence of serum as a initial step towards the exploring the possibility of these aptamers for therapeutic application. The biotinylated aptamer was coated followed by incubating the human serum for different time points (1, 20, 48 and 120 hours). FIG. 13 shows the ELISA results obtained for the stability of different aptamers tested in 100% and 20% serum. It could be observed that the aptamers were found to be highly stable in serum for about 120 hours. When the absorbance of the just aptamer (bars a) was compared with that of the aptamer treated for 48 and 120 hours with the 100% serum (bars b) and 20% serum (bars c), they are comparable without any major change (abs>1.6). Whereas in the negative sample (B03 heated at 95° C. for 48 hours), the absorbance at 450 nm is very low (abs<0.5) indicating that the continuous heating at 95° C. destabilizes the aptamer.

### Concluding Remarks:

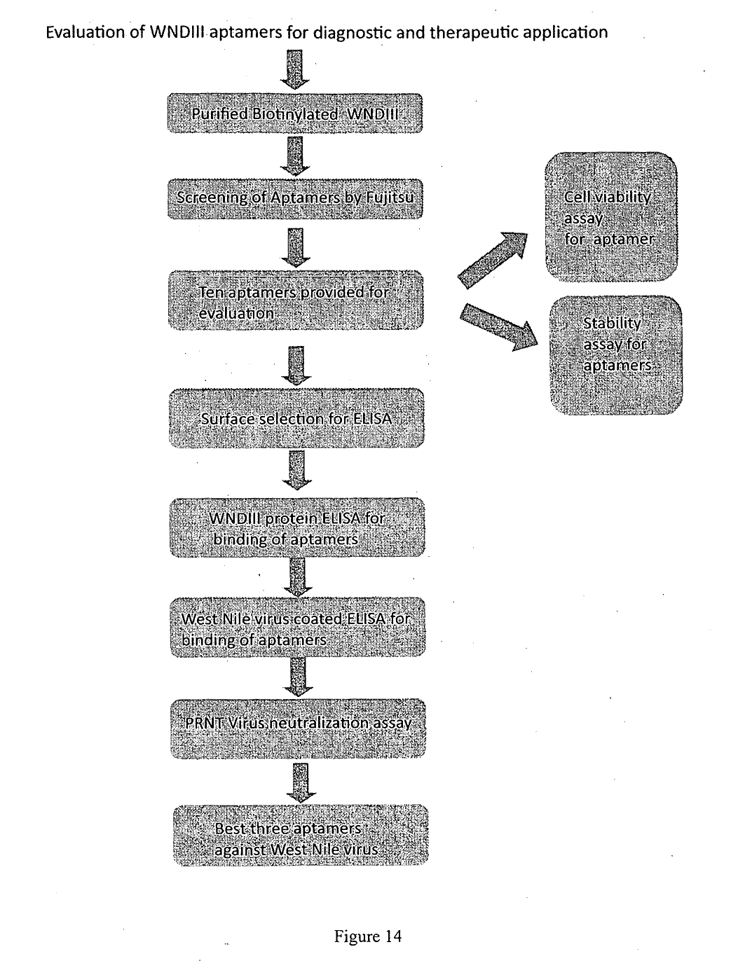

1. A new plasmid construct was designed for the production of biotinylated WNDIII for the first time. The biotin acceptor peptide (BAP) was engineered with the WNDIII gene for biotinylation. This construct can be utilized for both in vivo and in vitro biotinylation. In addition, the thrombin and enterokinase cleavage sites enable the removal of purification tags to yield the native protein after purification.
2. It was discovered that the BAP-WNDIII plasmid construct expressed in E. coli BL 21 (DE3) produces endogenously biotinylated protein. This endogenous biotinylation was confirmed by ELISA and Western Blot.
3. The endogenous biotinylation is not specific to WNDIII protein and is applicable to any protein-of-interest. This was also tested by cloning the BAP with the dengue virus capsid protein, and discovered that both the capsid and Dengue 2 envelope DIII protein were endogenously biotinylated via ELISA and Western blot.
4. A platform to obtain endogenously biotinylated, purified protein for biological applications like aptamer screening and studying protein-protein interaction, has been established.
5. The biotinylated proteins can be used in the development of diagnostics and therapeutics for Flaviviruses, and can be extended to other medically important pathogens.
6. As a proof-of-concept, the biotinylated WNDIII protein was used for screening and selection of modified aptamers by Fujitsu Laboratories.
7. Initial screening has resulted in the selection of ten aptamers from the library, which binds to WNDIII protein, by surface plasmon resonance. After the sequences were identified, Fujitsu scientists synthesized the ten aptamers (biotinylated and non-biotinylated aptamers) for evaluation.
8. The ten aptamers were evaluated against WNDIII protein and West Nile virus for binding and neutralization. The aptamers were also evaluated for any cytotoxic effect and their stabilities.
9. Initial evaluation was done for the surface of the ELISA plate. The Maxisorp plate was selected as a good surface for coating aptamers as well as the WNDIII protein for further evaluation.
10. Protein-coated enzyme linked modified aptamer sorbent assay for affinity screening revealed that aptamers B03, B79 and B99 bind to the WNDIII protein significantly when compared to other aptamers.
11. Virus-coated enzyme linked modified aptamer sorbent assay showed that aptamers B03, B79 and B99 bind specifically to the domain III of the native envelope protein present on the wildtype virus at even lower concentrations of aptamers.
12. Aptamers B03, B67, B73 and B99 bind significantly at concentrations higher than 3.3 nM to the Sarafend strain of WNV while aptamers B03, B66, B67, B73 and B79 bind significantly at the concentrations higher than 3.3 nM to the Kunjin strain. This indicated that the modified aptamers developed can be used for detection of different strains of West Nile viruses.
13. Based on the above evaluations, these aptamers can be developed into a diagnostic tool for West Nile virus detection, and also be extended to other flaviviruses including Dengue and Japanese encephalitis and other pathogens. Furthermore, the aptamers can also be used to develop molecular probes for the detection of virus in academic research.
14. Virus neutralization assay showed that 5 μM treatment of aptamers N03, N71, N79 and N99 resulted in about 30-35% neutralization, whereas the other aptamers showed less than 30% neutralization. When aptamer treatment concentration was at 10 μM, N03 and N99 showed neutralization higher than 50%. These results showed that N03 and N99 have the potential to be developed as a therapeutic against West Nile virus.
15. Viability assay results indicated that under the test conditions of 3.3 to 26 nM of aptamers treatment, the cells were viable for at least 60 hrs, similar to that of the untreated cells.
16. Stability assay showed that when aptamers were incubated for 5 days at room temperature and 37° C., the aptamers were stable and intact, and the bands could be detected by gel red.
17. Serum stability experiments showed that the aptamers are stable in 100% serum until 120 hours (5 days) at RT as detected by ELISA.
18. A complete platform for the production of BN-WNDIII protein and evaluation of the aptamers against the WNDIII protein is illustrated in FIGS. 3 and 14.
19. The three best candidate aptamers selected against WNV based on the evaluation are N03, N67 and N99 (Unlabeled aptamar) for therapeutic application and B03, B67 and B99 (Biotinylated aptamer) for diagnostic application. The sequences are listed in Table 3. These sequences will be further modified and evaluated for higher affinity.

TABLE 3
Aptamer sequences of the top three
anti-WNDIII A-Daptamers

| A-Daptamer | Aptamer | |
|---|---|---|
| ID | ID | sequence of variable region |
| anti- | WNDIII- | 5′-A_CfGkC_T_GwChC_A_CfAlA_GbT_ |
| WNDIII- | 003 | ChC_T_GwGbT_T_CyChC_T_Gw-3′ |
| 1-01 | | (based on modification of SEQ |
| | | ID No. 1) |
| anti- | WNDIII- | 5′-ChC_T_CyChC_AlA_A_CfAeT_GbT_ |
| WNDIII- | 067 | AsG_AsG_T_CyT_CyA_CfAeT_-3′ |
| 1-02 | | (based on modification of SEQ |
| | | ID No. 2) |
| anti- | WNDIII- | 5′-ChC_AlA_AeT_T_GwChC_GkC_AsG_ |
| WNDIII- | 099 | A_CfT_CyGbT_T_GwT_GwAlA_-3′ |
| 1-03 | | (based on modification of SEQ |
| | | ID No. 3) |

1. Backbone nucleotides are indicated in uppercase; A: Adenine, G: Guanine, C: Cytosine, T: Thymine
2. Functional groups of side chains are indicated in lowercase; b: Thiophene, e: Glutamic acid, f: Phenylalanine, h: Histidine, k: Lysine, l: Leucine, s: Serine, y: Tyrosine, w: Tryptophan
3. Native nucleotides are indicated with an underscore (_).

The following Example evaluates the stability and functionality of the modified aptamers for WNV DIII in the human and fetal bovine serum. Comparison studies with other modified and unmodified aptamers, and commercially available aptamer and antibody have also been carried out.

### Example 2: Evaluation of Stability and Functionality of WNDIII Aptamers in Serum

### Stability of Aptamers in Human Serum.

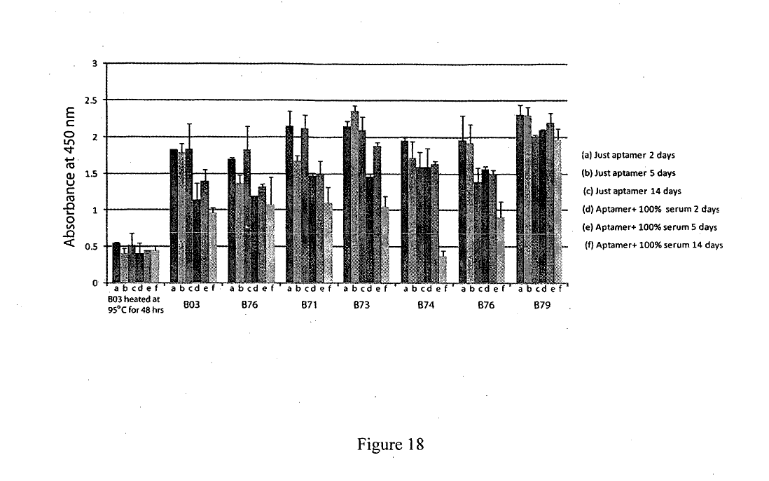

In order to test the stability of the modified aptamers by ELISA, biotinylated WNDIII aptamers (B03, B66, B71, B73, B74, B76 and B79 obtained from, Apta Biosciences Pte Ltd www.aptabiosciences.com, 31 Biopolis Way, #02-25 Nanos, Singapore 138669, Phone: +65-3109-0178, Fax: +65-6779-6584, Mobile: +65-9184-7323) formerly known as Fujitsu Biolaboratories) were coated on a maxisorp plate (40 ng/well) followed by incubation with human serum for different durations. If the aptamer was unstable, it would degrade and be removed during washing. Otherwise, the stable modified aptamer would remain bound to the maxisorp plate. The presence of the biotinylated aptamer would then be detected by a streptavidin-HRP conjugate, thereby resulting in TMB substrate conversion. The serum stability of the modified aptamers was monitored for up to 14 days, and was found to vary between 50% and 90% when compared to their respective serum-free controls as shown in FIG. 18. The negative control involved modified aptamer B03 heated at 95° C. for 48 hours, which showed that the modified aptamers were unstable under prolonged heating.

Based on results from the stability studies of aptamers in human serum, the modified aptamers could be classified into Type 1: Moderately stable (B74), Type 2: Highly stable (B03, B66, B71, B73, B76 and Type 3: Very highly stable—(B79). This implied that the backbone of modified aptamer B79 can be used as the starting template to generate highly stable aptamers in the future. Although modified aptamer B79 was shown to have the highest stability, as can be seen from FIG. 18, modified aptamers B03 and B99 were selected for further studies because they were among the top three modified aptamers with the best binding and virus neutralization, and had the potential to be developed as a diagnostic reagent or therapeutic candidate. Nonetheless, this experiment showed that the modified aptamers were much more stable in serum than other aptamers (Kaur et al., 2013, Peng et al., 2007), and could be potential therapeutics with long physiological half-lives.

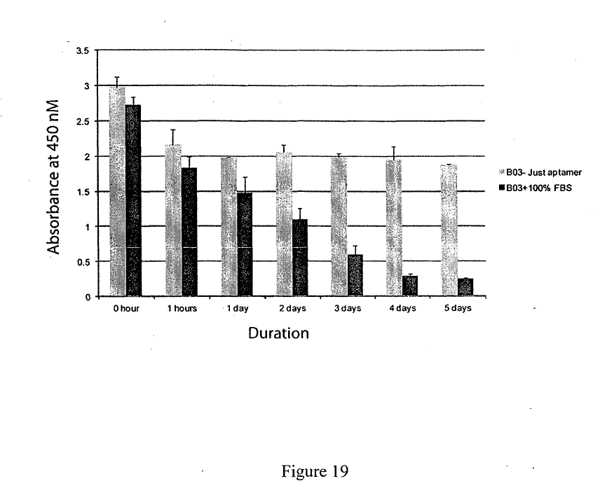

Comparison on the stability of modified aptamer B03 in fetal bovine serum (FBS) for 5 days was also made. FIG. 19 shows that the stability of modified aptamer B03 decreased with time in FBS. One possible reason might be due to the presence of destabilizing agents such as bovine nucleases in FBS. Previous studies have shown that the unmodified aptamers of the B cell receptors has a half-life of 1 hour in serum whereas modification with locked nucleic acids (LNA) increased the half-life to ˜9 hours (Mallikaratchy et al., 2010). Modified aptamers showed nuclease resistance up to 14 days in 100% human serum and 4 days in 100% FBS.

### Functionality Test of Modified Aptamers in Human Serum

### Binding of Aptamers to WNV DIII and WNV in Human and Fetal Bovine Serum.

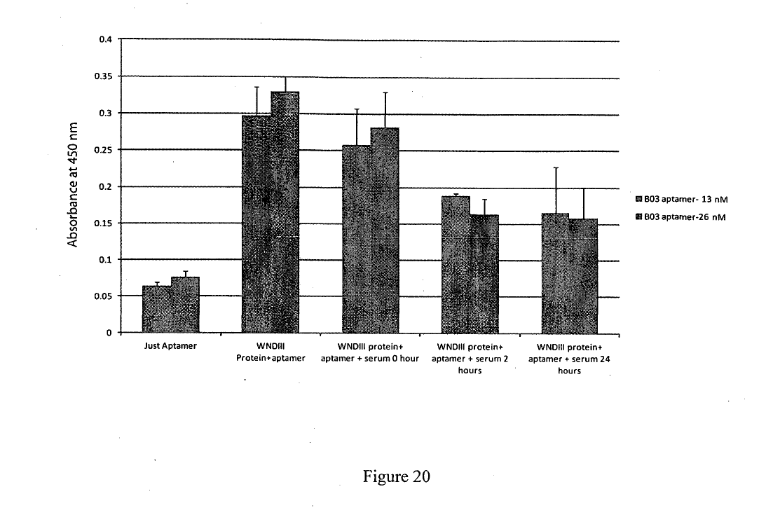

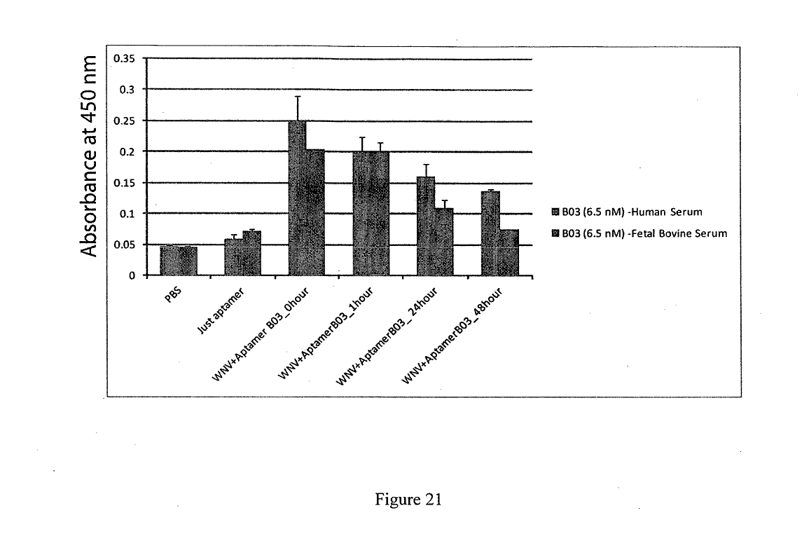

Using ELISA as the platform, maxisorp plates were coated with either WNV DIII protein or WNV. Different concentrations of biotinylated WNV DIII modified aptamer B03 was then added and incubated for 2 hours to allow the modified aptamer to bind to the target. Neat human serum or FBS was subsequently added and incubated for different durations. After incubation, the presence of modified aptamers was probed with streptavidin-HRP conjugate, followed by TMB substrate development. FIG. 20 shows that when the maxisorp plate coated with WNV DIII protein was used, it was found that for both aptamer concentrations tested, modified aptamer B03 was able to bind to the target protein in human serum for up to 24 hours. Similarly, modified aptamer B03 was able to bind to wildtype WNV in human serum for up to 48 hours as seen from FIG. 21. In contrast, this ability to bind to virus was gradually reduced in FBS. This could again be due to the instability of the aptamer in FBS.

### Evaluation of Unmodified Aptamers for Stability and Functionality by ELISA

Polynucleotides corresponding to the DNA backbone of the WNV DIII modified aptamers B03 and B99 (i.e. unmodified aptamers) were synthesized (Sigma Aldrich, USA) for comparison with the modified aptamers (which have peptide side chains) in terms of stability and functionality. The nucleotide sequences corresponding to the DNA backbone of the WNV DIII modified aptamers B03 and B99 are listed below.

| | |
|---|---|
| BN-B03-DNA | BN-5′GAAGGTGAAGGTCGGCTGAAGCATTAG |
| | ACCTAAGCACGCTGCCACAAGTCCTGGTTCCC |
| | TGGCTTAGGTCTAATGC ACCATCATCACCAT |
| | CTTC 3′(SEQ ID No. 11) |
| BN-B99-DNA | BN-5′GAAGGTGAAGGTCGGCTGAAGCATCA |
| | GACCTAAGCCCAAATTGCCGCAGACTCGTTGT |
| | GAAGCTTAGGTCTAATGC ACCATCATCACCA |
| | TCTTC-3′ (SEQ ID No. 12) |

For the stability comparison study, known amounts of unmodified DNA aptamers were incubated at room temperature (RT) for varying durations in human serum or FBS. Their stability was then determined through detection using streptavidin-HRP conjugate in ELISA.

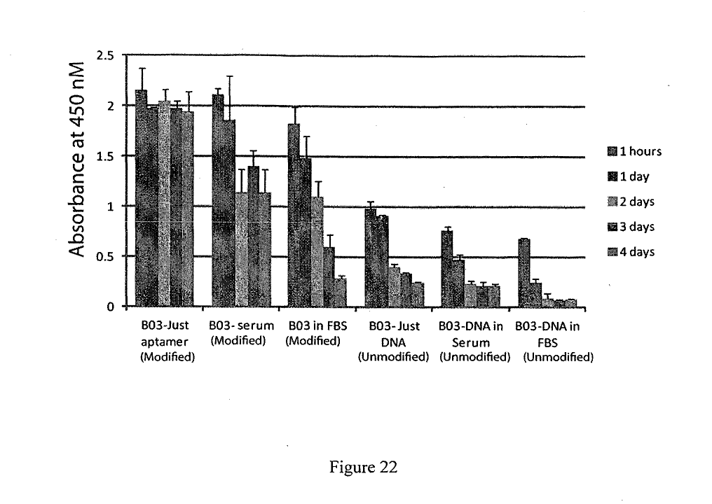

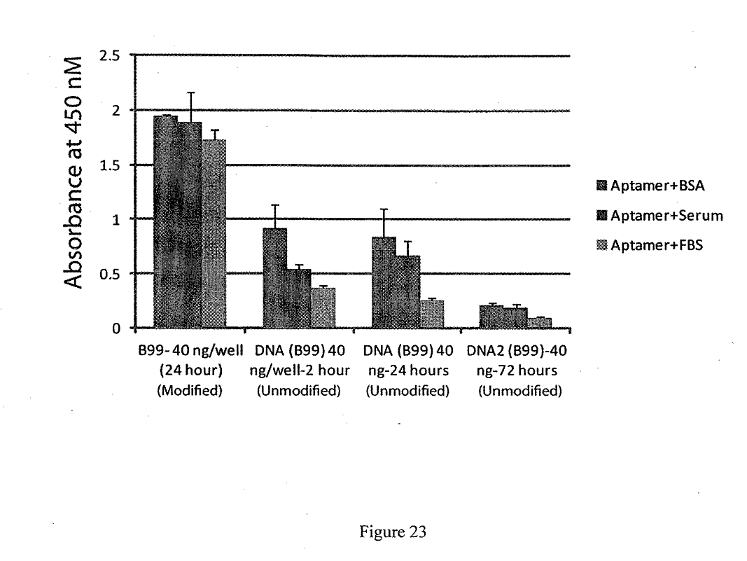

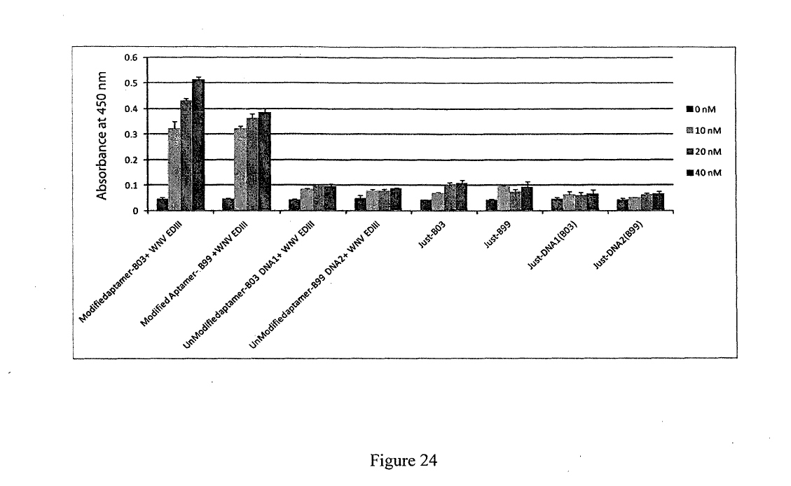

Based on the stability study as shown in FIGS. 22 and 23, it could be concluded that WNDIII modified aptamers B03 (see FIG. 22) and B99 (see FIG. 23) were very stable in human serum and moderately stable in FBS. The stability of the corresponding unmodified DNA aptamers corresponding to the nucleotide sequence of aptamers B03 and B99 were much lower in human serum and FBS. This indicated that additional stability was conferred by the side-chain modifications in the modified aptamers. Similarly, when the functionality of the WNV DIII side-chain modified aptamers B03 and B99, and their unmodified DNA counterparts were tested, it was observed that the unmodified DNA aptamers were unable to bind to the target protein, as can be seen from FIG. 24.

Comparison of Aptamer Binding with WNV DIII Commercial Antibody

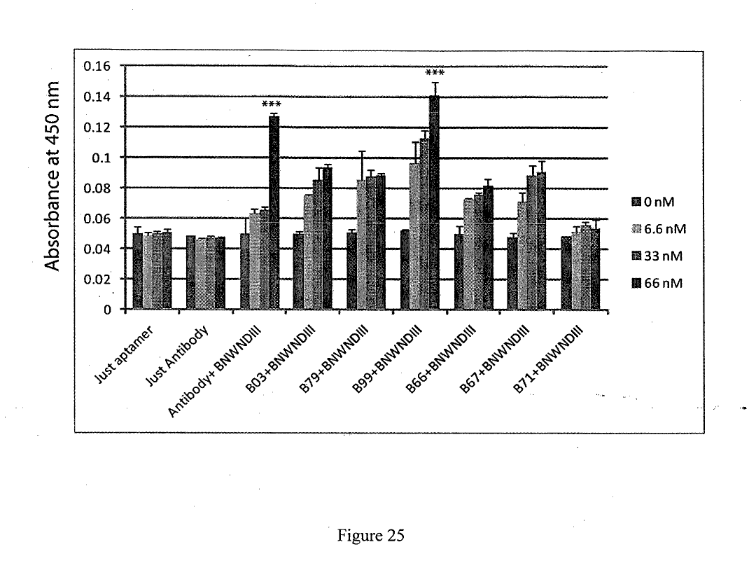

Using the ELISA platform, the same concentration (33 nM) of aptamers (B03, B79, B99, B66, B67, B71) and WNV-specific antibody (Millipore MAB8151) were coated onto a maxisorp plate to capture biotinylated WNV DIII protein. FIG. 25 shows that both the aptamers and antibody were able to capture the WNV DIII protein. Modified aptamer B99 had the strongest binding and was comparable to the antibody. This was followed by modified aptamers B03, B79, B66 B67 and B71.

### Concluding Remarks:

1. Stability of modified aptamers in human serum varies between 50% and 90% for up to 14 days, and varies between individual aptamer. The modified aptamers can be classified according to their stability in human serum into type 1: Moderately stable (B74), type 2: Highly stable (B03, B66, B71, B73 and B76) and type 3: Very highly stable (B79).
2. Modified aptamer (B03) was able to bind to WNV DIII protein and wildtype WNV for up to 24 and 48 hours in human serum, respectively.
3. The stability and functionality results indicated that modified aptamers were functional in human serum, a property essential for modified aptamers to be developed as a diagnostic tool or therapeutic candidate.
4. Comparison studies on the stability between side-chain modified WNV DIII aptamers B03 and B99, and their unmodified DNA counterparts indicated that modified aptamers B03 and B99 were highly stable whereas their unmodified DNA counterparts became unstable after 24 hours of incubation in human serum and FBS.
5. Comparison studies on the functionality between side-chain modified WNV DIII aptamers B03 and B99, and their unmodified DNA counterparts indicated that modified aptamers B03 and B99 could bind to WNV DIII protein whereas their unmodified DNA counterparts could not.
6. Both the modified aptamers and antibody were able to bind WNV DIII protein at the same concentration. Binding of modified aptamer B99 to WNV DIII protein was the strongest and was comparable to that of the antibody, followed by modified aptamers B03, B79, B66 and B67.

### Example 3: Evaluation of Dengue Virus Serotype 2 (DENV2) Modified Aptamers

The following Example evaluates the binding characteristics of a separate set of selected modified aptamers (generated by Adaptamer Solutions, www.aptabiosciences.com, Apta Biosciences Pte Ltd, 31 Biopolis Way, #02-25 Nanos, Singapore 138669, Phone: +65-3109-0178, Fax: +65-6779-6584, Mobile: +65-9184-7323) against purified DENV2 DIII protein and the native envelope protein on wildtype DENV. The best aptamer which can be utilized for diagnostic and therapeutic applications was then identified. Ten potential aptamer candidates against DENV2 DIII protein were evaluated and the results are also discussed.

### Materials and Methods

### Cloning and Expression of DENV1-4 Biotinylated Recombinant Envelope Domain III (DENV1-4 BN-rEDIII) Protein

Overlapping Extension-Polymerase Chain Reaction (OE-PCR).

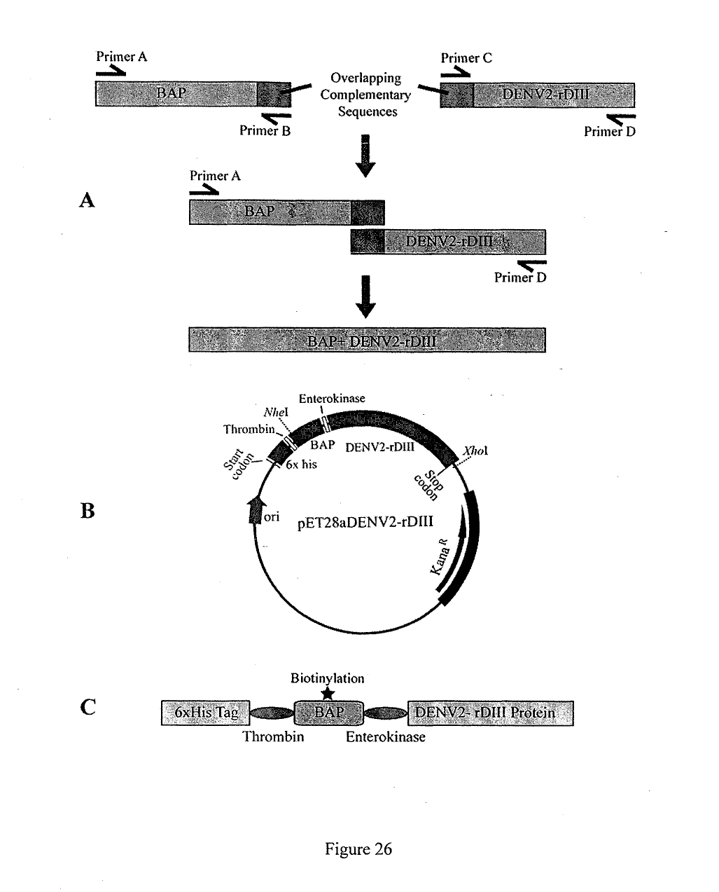

Two fragments were used in the cloning of DENV1-4 BN-rEDIII protein. The biotin acceptor peptide (BAP) (Fragment 1) was synthesized chemically. Domain III of the envelope glycoprotein (Fragment 2) of each DENV serotypes was derived from the cDNA of DENV1-4, respectively. FIG. 26 illustrates the steps involved in the construction of the DENV2 BN-rEDIII plasmid. A similar strategy was also followed to obtain DENV1, 3 and 4 BN-rEDIII proteins for downstream aptamer screening. The list of primers used in OE-PCR is shown in Table 4.

TABLE 4
The list of forward and reverse primers used in
OE-PCR to join Fragment 1 (BAP) and Fragment 2
(DIII gene) for all four DENV serotypes.

| | |
|---|---|
| DENV1 | Primer A (BAP Forward): 5′CTAGCTAGCTCCGG |
| BN-rEDIII | CCTGAACGAC NheI (SEQ ID No. 13) |
| Primers for | Primer B (BAP Reverse): |
| Bacterial | 5′ATATGACATCCCTTTTAAGCTCTTGTCGTCGTC |
| expression | (SEQ ID No. 14) |
| | Primer C (D1-DIII Forward): |
| | 5′GACGACGACAAGAGCTTAAAAGGGATGTCATAT |
| | (SEQ ID No. 15) |
| | Primer D (D1-DIII Reverse): 5′CCGCTCGAGT |
| | TAGCTTCCCTTCTTGAA XhoI (SEQ ID No. 16) |
| DENV2 | Primer A (BAP Forward): 5′CTAGCTAGCTCCGG |
| BN-rEDIII | CCTGAACGAC NheI (SEQ ID No. 17) |
| Primers for | Primer B (BAP Reverse): |
| Bacterial | 5′GTATGACATTCCTTTGAGGCTCTTGTCGTCGTC |
| expression | (SEQ ID No. 18) |
| | Primer C (D2-DIII Forward): |
| | 5′GACGACGACAAGAGCCTCAAAGGAATGTCATAC |
| | (SEQ ID No. 19) |
| | Primer D (D2-DIII Reverse): 5′CCGCTCGAGT |
| | TAACTTCCTTTCTT XhoI (SEQ ID No. 20) |
| DENV3 | Primer A (BAP Forward): 5′CTAGCTAGCTCCGG |
| BN-rEDIII | CCTGAACGAC NheI (SEQ ID No. 21) |
| Primers for | Primer B (BAP Reverse): |
| Bacterial | 5′ATAGCTCATCCCCTTGAGGCTCTTGTCGTCGTC |
| expression | (SEQ ID No. 22) |
| | Primer C (D3-DIII Forward): |
| | 5′GACGACGACAAGAGCCTCAAGGGGATGAGCTAT |
| | (SEQ ID No. 23) |
| | Primer D (D3-DIII Reverse): |
| | 5′CCGCTCGAGTTAGCTCCCCTTCTTGTA XhoI |
| | (SEQ ID No. 24) |
| DENV4 | Primer A (BAP Forward): 5′CTAGCTAGCTCCGG |
| BN-rEDIII | CCTGAACGAC NheI (SEQ ID No. 25) |
| Primers for | Primer B (BAP Reverse): |
| Bacterial | 5′GTATGACATTCCCTTGATGCTCTTGTCGTCGTC |
| expression | (SEQ ID No. 26) |
| | Primer C (D4-DIII Forward): |
| | 5′GACGACGACAAGAGCATCAAGGGAATGTCATAC |
| | (SEQ ID No. 27) |
| | Primer D (D4-DIII Reverse): |
| | 5′CCGCTCGAGTTAACTCCCTTTCCTGAA XhoI |
| | (SEQ ID No. 28) |

Protein Expression and Extraction.

pET28a-DENV2 BN-rEDIII plasmid was transformed into BL-21-DE3 expression competent cells (Agilent Technologies, USA) and grown in Luria-Bertani (LB) agar containing 30 μg/ml kanamycin. Selected clones were cultured in 1 L LB broth (30 μg/ml kanamycin) at 30° C. until an OD₆₀₀ of 0.6. Expression of DENV2 BN-rEDIII protein was induced with 1 mM isopropyl β-D-thiogalactoside (IPTG) for 6 hours. Bacterial cells were pelleted down with centrifugation at 8,000 rpm for 15 min at 4° C. The protein expressed was targeted to inclusion bodies (IB). IBs were isolated in the subsequent steps. The bacterial cell pellet was first resuspended in lysis buffer (20 mM Tris pH 8.0, 500 mM NaCl, 10 mM imidazole), followed by sonication in ice bath (10 min, 10 Amp). The lysate was then centrifuged at 12,000 rpm for 15 min at 4° C. to obtain a small white translucent pellet of inclusion body. The inclusion body pellet was then washed with the same lysis buffer, incubated in extraction buffer (8 M urea, 20 mM Tris, 300 mM NaCl, 10 mM imidazole, pH 8.0) at room temperature for 30 min, and its extract clarified by centrifugation at 13,500 rpm for 20 min.

Immobilised Metal Ion Affinity Chromatography (IMAC) Purification of BN-rEDIII Protein.

The inclusion body extract containing DENV2 BN-rEDIII protein was incubated with nickel-nitrilotriacetic acid (Ni-NTA) resin (Bio-Rad, USA) for binding in a chromatography column overnight at 4° C. Five column volume of wash buffer (8 M urea, 20 mM Tris, 300 mM NaCl, 20 mM imidazole, pH 8.0) was used to remove non-specific binding proteins. BN-D2DIII protein was then eluted out with elution buffer (8 M urea, 20 mM Tris, 300 mM NaCl, 500 mM Imidazole, pH 8.0) in eight 1.5-ml fractions. All the eluates were pooled into a SnakeSkin dialysis membrane tubing (Thermo Scientific, USA) and 0.05% of Tween-20 was added to the samples. The dialysis tubing was incubated in 4 M urea for 6-12 hrs at 4° C., and the urea diluted stepwise to 0.5M. The refolded DENV2 BN-rEDIII protein was finally collected from the dialysis tubing and injected into a FPLC machine to be further purified via size-exclusion chromatography into PBS. DENV1, 3 and 4 BN-rEDIII proteins were also purified in a similar manner.

Protein Identity Analysis.

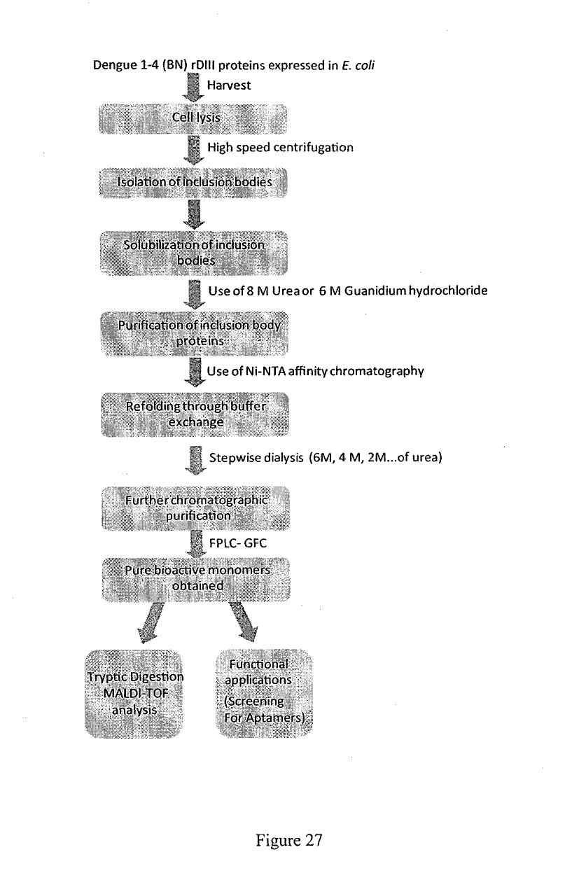

The flow through, wash, and eluates from the IMAC purification were analyzed by SDS-PAGE and Western blot. 12% Tris-tricine polyacrylamide denaturing gel was used to separate proteins and was subsequently stained with Coomassie blue for protein detection. For Western blotting, proteins were transferred from the polyacrylamide gel onto a PVDF membrane using iBlot® Dry Blotting System (Life Technologies, USA). Blocking was done with 5% BSA overnight in 4° C. The membrane was then incubated with streptavidin conjugated-HRP to detect for DENV BN-rEDIII for 2 hours at room temperature. The membrane was washed thoroughly with 1×PBST for 1 hour at room temperature and developed with SuperSignal® West Pico chemiluminescent substrate (Thermo Scientific, USA). A schematic flowchart representing the expression, purification and evaluation of recombinant purified DENV1-4 BN-rEDIII proteins is shown in FIG. 27.

Protein-Coated Enzyme-Linked Modified Aptamer Sorbent Assay (ELMASA) for Affinity screening.

100 ng of purified non-biotinylated DENV2 rEDIII protein was coated onto each well of a maxisorp plate overnight at 4° C. On the following day, the ELISA plate was washed three times with Phosphate-buffered saline (PBS) and incubated for 1 hour with different concentrations (1 to 32 nM/well) of biotinylated (DENV) aptamers solubilized in RNase free TE buffer (Invitrogen) in triplicates. Blocking with 4% BSA in PBS was then carried out overnight, followed by washing with PBS. 1:2000 (v/v) dilution of streptavidin-HRP enzyme conjugate (Millipore) was subsequently added and the plate was incubated for 1 hour. The plate was washed 6 times with 1×PBST, before 50 μl of tetramethyl benzidine (TMB) substrate solution was added and incubated for 15 min at room temperature. Finally, 50 μl of 0.5 M H₂SO₄ solution was added to stop the reaction and absorbance was measured immediately at 450 nm.

Virus-Coated ELMASA.

Instead of using DENV2 rEDIII protein, 1,000 PFU of DENV2 wildtype virus was coated onto the ELISA plate and incubated overnight at 4° C. The wells were washed with 1×PBST followed by blocking with 4% BSA. Following this step, the wells were incubated with different concentrations (1 to 32 nM) of biotinylated aptamers (1-10) for 1 hour. 1:2000 (v/v) dilution of streptavidin-HRP enzyme conjugate was then added and the rest of the experiment was performed as described in the protein-coated ELMASA above. All procedures were carried out in a class 2 Biological Safety Cabinet (BSC).

Virus Blocking Assay.

BHK cells were seeded in a 24-well plate overnight at 50000 cells/well. 50 μl of 2 μM aptamers solubilized in RNase-free TE buffer (Invitrogen) were added to 50 PFU/50 μl DENV2 in triplicates. The mixture was incubated for 1.5 hrs for binding (final aptamer working concentration is 1 μM/well). A negative control was set up similarly without any virus. Following which, growth medium was removed from the 24-well plate, and the cell monolayer in each well was washed with RPMI containing 2% FCS and infected with the 100 μl of aptamer-virus mixture. The plate was incubated at 37° C. and 5% CO₂ for 1 hour, with constant rocking at 15-min interval. The inoculum was removed, the cell monolayer washed with RPMI containing 2% FCS, and 1 ml of CMC overlay medium wad added to each well. The plate was incubated at 37° C. and 5% CO₂ for 4.5 days until plaques were formed. The remaining cells were finally stained with crystal violet and the unstained plaques were counted.

### Results

### Construction of DENV1-4 BN-rEDIII Plasmids

To obtain DENV1-4 BN-rEDIII proteins for aptamer screening, new expression plasmids were designed by engineering in a biotinylation acceptor peptide (BAP), followed by an enterokinase cleavage site, at the N-terminus of the DENV1-4 envelope DIII gene. The DNA sequence corresponding to the BAP was chemically synthesized (Kaur et al., 2013), whereas the DENV1-4 envelope DIII DNA sequences were derived from the cDNA of DENV1-4, respectively. The BAP sequence with the enterokinase cleavage site was linked upstream of DIII through overlapping extension PCR (OE-PCR) as illustrated in FIG. 26A. The final PCR product and pET28a vector were double-digested with NheI and XhoI restriction enzymes and the recombinant gene ligated into the digested plasmid, which contained a 6×His tag upstream of the multiple cloning site. Thus, recombinant BAP-containing DENV1-4 rEDIII proteins each had 2 tags at the N-terminus, namely the 6×His tag (for affinity purification) and biotin (to bind to streptavidin). Each of them also contained two enzyme (thrombin and enterokinase) cleavage sites (see FIGS. 26B & 26C). This engineered construct was then transformed into E. coli TOP 10 cells and positive clones were verified by colony PCR, restriction digestion and DNA sequencing. Biotinylation of recombinant BAP-contain DENV1-4 rEDIII proteins could thus be performed both in vivo and in vitro. In addition, the thrombin and enterokinase cleavage sites enabled removal of the 6×His tag with or without the biotinylated BAP after purification. This allowed the purified rEDIII proteins to be used in other downstream applications, such as the selection of protein interacting partners and/or aptamers from protein and/or aptamer library.

### Expression and Purification of DENV1-4 BN-rEDIII Proteins

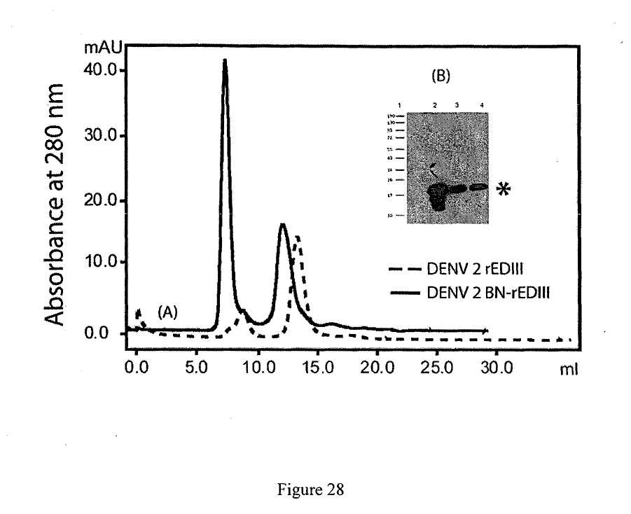

The DENV1-4 BN-rEDIII proteins were expressed in E. coli BL21 (DE3). After DENV1-4 BN-rEDIII protein expression was confirmed via Western blotting using an anti-His antibody, expression was scaled up to produce large amounts of DENV1-4 BN-rEDIII proteins. Crude protein was extracted from the inclusion bodies and subjected to IMAC affinity purification, refolding, and size exclusion chromatography as explained in Materials and Methods. The representative FPLC-SEC profile for DENV2 BN-rEDIII protein is shown in FIG. 28. For comparison, the trace corresponding to unbiotinylated DENV 2 rEDIII protein was superimposed. Western blotting using streptavidin-HRP confirmed the presence of biotin on DENV1-4 BN-rEDIII and their purities as shown in FIG. 28 (B). Only a single band was detected in the purified fractions after FPLC (Lanes 3 and 4), whereas multiple bands were detected in the IMAC eluate, prior to SEC purification (Lane 2). 1 mg of purified rEDIII protein was purified from 1 L of bacteria culture. The identities of the purified DENV1-4 BN-rEDIII proteins were further confirmed by in-gel tryptic digestion and peptide mass fingerprinting.

### Aptamer Screening:

### Aptamer Designing and Synthesis:

### Identification of DENV2 BN-rEDIII Protein-Binding Modified Aptamer Candidates

DENV2 BN-rEDIII protein immobilized on monomeric avidin-agarose resin was incubated with a library solution of modified aptamers. The resin was then washed repeatedly to remove weakly bound modified aptamers before the modified aptamer: DENV2 BN-rEDIII complexes were eluted from the resin using a biotin solution. The eluted complexes were treated with alkali to remove the side chains and liberate the DNA aptamer backbone for PCR, sequencing, and subsequent cloning to allow determination of the DNA sequence of the bound aptamers. DNA sequences of 136 DENV2 BN-rEDIII modified aptamer candidates were obtained and these modified aptamers were synthesized by a DNA synthesizer. Screening of DENV2 BN-rEDIII modified aptamer candidates was repeated by applying them to DENV2 BN-rEDIII protein immobilized on a CM5 Biacore sensor chip by amine-coupling. The top 10 DENV2 BN-rEDIII modified aptamer candidates were selected for further analysis.

SPR Analysis Using DENV2 rEDIII Protein:

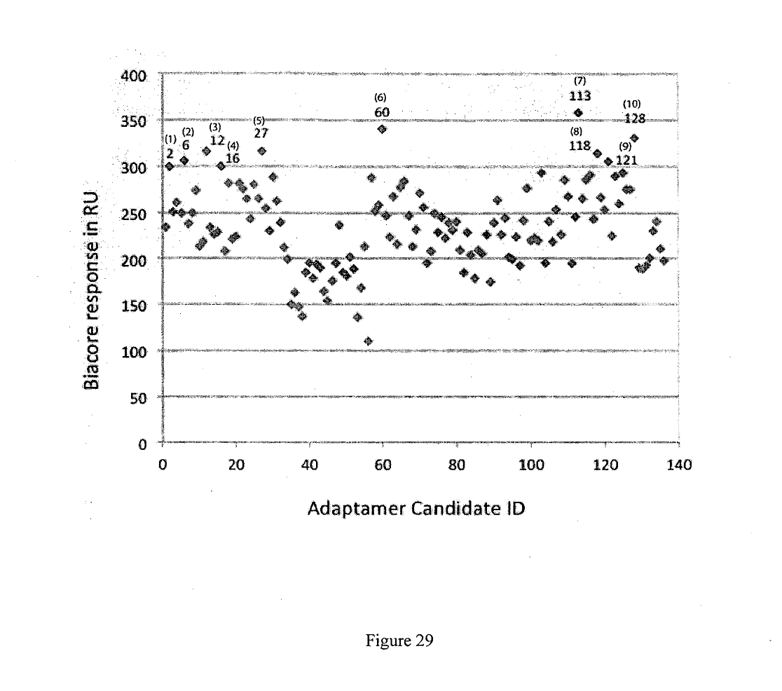

For K_{D} measurement, each of the ten DENV2 BN-rEDIII modified aptamer candidates was biotinylated and immobilized on a Biacore SA chip separately. Their individual K_{D} was determined for various concentrations of DENV2 rEDIII protein in MES buffer at pH 5.5 (see FIG. 29 and Table 5). The ten aptamers received from Adaptamer Solutions for validation against DENV2 EDIII are biotinylated modified aptamers B002, B006, B012, B016, B027, B060, B113, B118, B121 and B128.

TABLE 5
List of aptamers chosen for further evaluation after
measurement of their affinities using SPR.

| | Screening | K_{D} at pH |
|---|---|---|
| Aptamer ID | (RU) | 5.5 (nM) |
| D2ED3-002 | 251 | 53.1 |
| D2ED3-006 | 306 | 16.8 |
| D2ED3-012 | 316 | 18.3 |
| D2ED3-016 | 300 | 23.0 |
| D2ED3-027 | 317 | 33.1 |
| D2ED3-060 | 341 | 27.7 |
| D2ED3-113 | 358 | 7.1 |
| D2ED3-118 | 314 | 15.8 |
| D2ED3-121 | 305 | 13.9 |
| D2ED3-128 | 331 | 21.2 |

### DENV2 BN-rEDIII Coated ELMASA for Affinity Screening of Modified Aptamers.

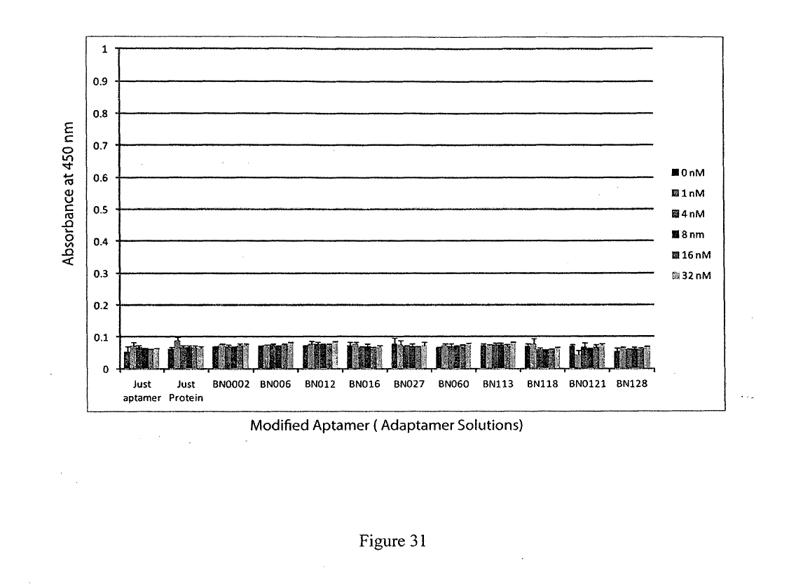

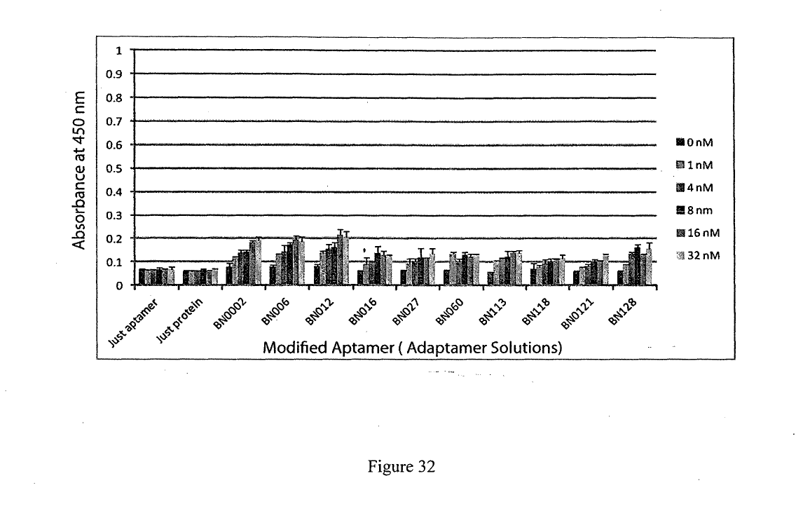

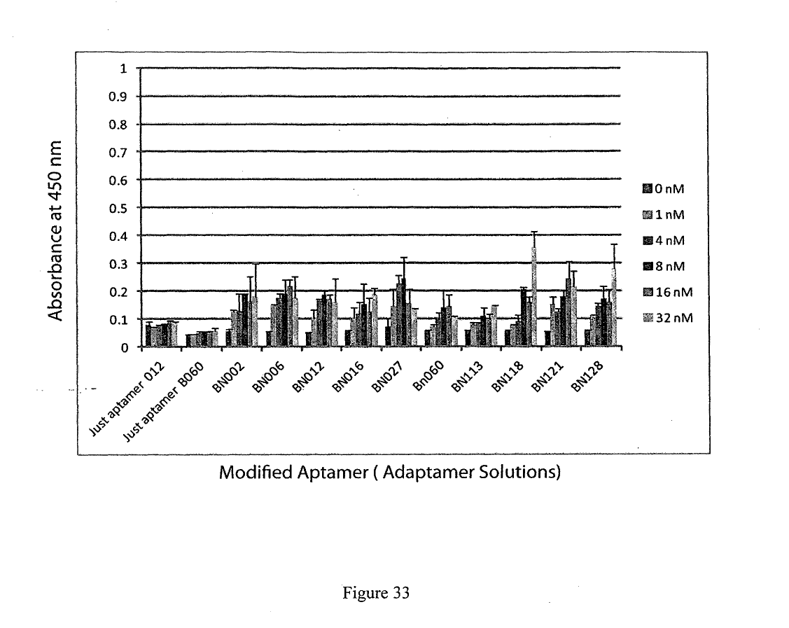

In order to evaluate the binding of the 10 selected modified aptamers to DENV2 rEDIII protein, DENV2 rEDIII protein coated ELMASA was carried out using biotinylated modified aptamers of various concentrations (0 to 32 nM). It was observed that modified aptamers B002, B006, B027 and B128 bound most efficiently to DENV2 rEDIII protein although modified aptamers B012, B060, B113, B118 and B121 also bound significantly to the DENV2 rEDIII proteins at all concentrations tested. The binding of the modified aptamers against rEDIII protein of DENV1, 3 and 4 were evaluated, and the results were shown in FIGS. 31, 32 and 33, respectively. For all 10 modified aptamers, there was minimal binding to the rEDIII proteins of DENV1, 3 and 4. This result implied that the modified aptamers bound specifically to the DENV2 rEDIII protein.

### Virus-Coated ELMASA.

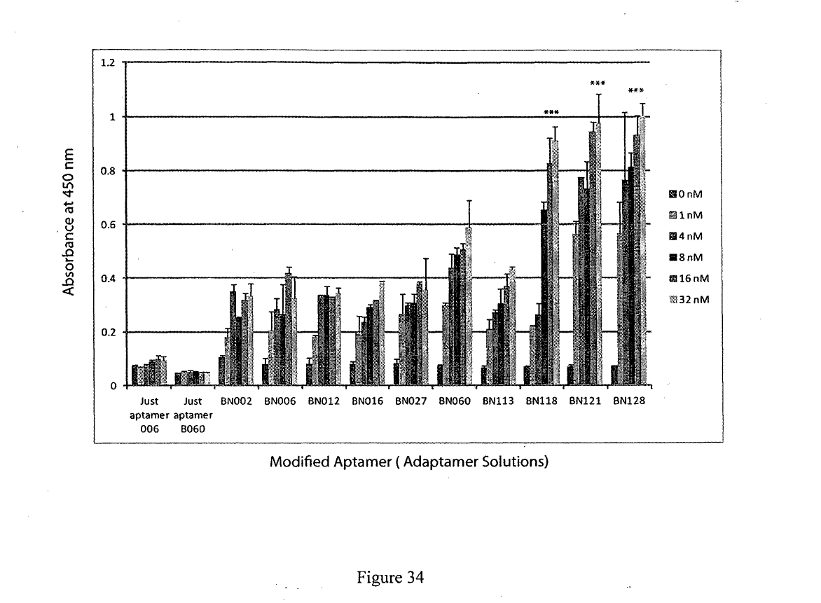

Binding of the modified aptamers to purified DENV2 rEDIII protein was further confirmed using wildtype virus. DENV2 (1000 PFU/well) was coated on the ELISA plate overnight, followed by incubation with different concentrations of aptamers. It was still observed that modified aptamers B060, B118, B121 and B128 bound significantly to DENV2 as compared with the control (FIG. 34). This implied that these modified aptamers could bind specifically to the native envelope domain III on wildtype DENV2, and that the modified aptamers can be used for the detection and differentiation of different DENV serotypes, a feat only currently possible via PCR.

### Neutralization of DENV2 by Modified Aptamers

After establishing that the modified aptamers were able to bind to purified DENV2 rEDIII protein and native envelope DIII protein on wildtype DENV2, their ability to neutralize DENV2 was evaluated. Prior incubation of viruses with different concentrations of modified aptamers, followed by infection of BM cells was carried out. There was a reduction in the number of virus-induced plaques when DENV2 was pretreated with 1 μM of modified aptamer. The results showed that pretreatment with 1 μM of modified aptamers B060 and B118 resulted in more than 60% neutralization, whereas neutralization by the other modified aptamers varied between 40% and 58%. Thus, modified aptamers B060 and B118 had the potential to be developed into therapeutics against DENV2.

Cross Reactivity of DENV2 DIII Modified Aptamers with Other Flavivirus Envelope Protein:

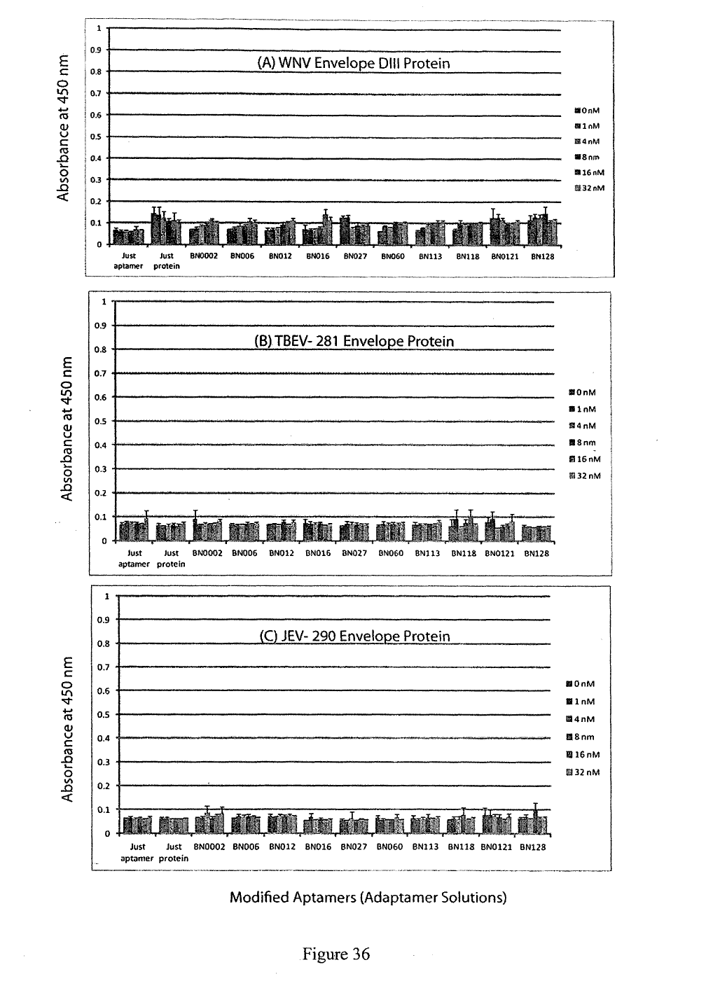

In order to evaluate potential non-specific and cross-reactive binding of the modified aptamers to other flavivirus envelope protein, protein coated ELMASA was performed using the envelope or DIII proteins of West Nile virus (WNV), tick-borne encephalitis virus (TBEV) (ProSpecbio, USA) and Japanese Encephalitis Virus (JEV) (ProSpecbio, USA). No significant binding to the envelope or DIII proteins of all three viruses above was detected at all the modified aptamer concentrations tested (see FIG. 36; Panel A: WNV envelope DIII, Panel B: TBEV-281 envelope protein, Panel C: JEV-290 envelope protein).

TBE-281:

Tick-borne encephalitis is caused by tick-borne encephalitis virus (TBEV), a member of the virus family Flaviviridae. TBE-281 is the E. coli derived recombinant protein comprising residues 95 to 229 of the Tick-borne Encephalitis Virus envelope glycoprotein.

JEV-290:

Japanese encephalitis previously known as Japanese B encephalitis is a virus from the virus family Flaviviridae. It is closely related to WNV and St. Louis encephalitis virus. JEV-290 protein is the 50-kDa full length Japanese Encephalitis virus envelope protein expressed in E. coli and is fused to a 6× histidine tag.

Comparison of Binding for the DENV2 DIII Modified Aptamers of the Present Invention and Other Commercial Aptamer to DENV2 rEDIII Protein.

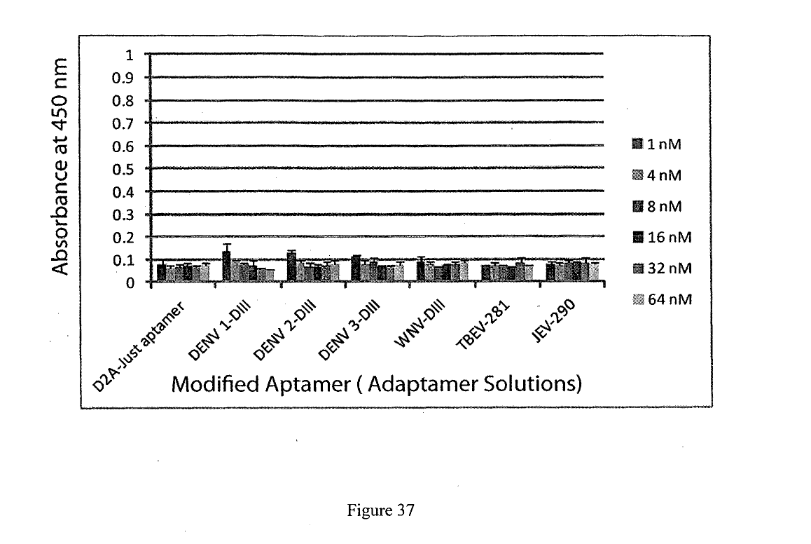

The functionality of the DENV2 DIII modified aptamers of the present invention was compared to that of commercially available aptamers against DENV2 DIII (D2A) (OTC Biotech, USA). The commercial aptamer was evaluated in a similar manner as the DENV2 DIII modified aptamers. As illustrated in FIG. 37, the commercial aptamer was unable to bind with all the target proteins at the tested concentrations. In comparison, modified aptamer B128 showed very high absorbance in ELISA, indicating significant binding to DENV2 rEDIII protein.

### Concluding Remarks:

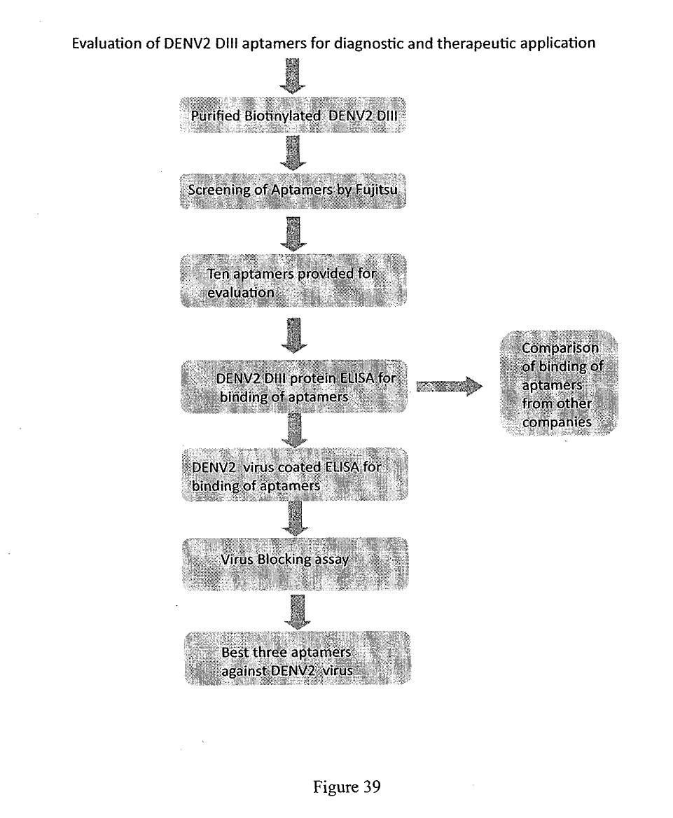

1. A plasmid construct was designed for production of biotinylated DENV1-4 rEDIII proteins for the screening of modified aptamers. A biotin acceptor peptide (BAP) has been engineered into the genes of DENV1-4 rEDIII for biotinylation. This construct can be utilized for both in vivo and in vitro biotinylation. The insertions of thrombin and enterokinase cleavage sites further enable the removal of tags to yield native proteins after purification.
2. A platform has been established to obtain biotinylated, purified DENV 1-4 DIII for applications such as aptamer screening and studying of protein-protein interactions.
3. Biotinylated DENV2 rEDIII protein was used for screening and selection of modified aptamers by Adaptamer Solutions.
4. Initial screening has resulted in the selection of ten modified aptamers, which bind to DENV2 rEDIII protein, by surface plasmon resonance from the library. After the sequences were identified, Adaptamer Solutions scientists synthesized the ten modified aptamers (biotinylated aptamers) for evaluation.
5. The ten biotinylated modified aptamers were evaluated against DENV2 rEDIII protein and DENV2 for binding and neutralization, respectively.
6. Protein-coated ELMASA for modified aptamer affinity screening revealed that modified aptamers B002, B118 and B128 bound to DENV2 rEDIII protein specifically.
7. Virus-coated ELMASA showed that modified aptamers B118, B121 and B128 bound specifically to the native envelope protein present on wildtype DENV2 even at low concentrations. Based on the above evaluations, these aptamers can be developed into a diagnostic tool for DENV detection. These aptamers can also be developed into molecular probes for the detection of virus for academic research.
8. Virus neutralization assay showed that treatment using 1 μM of modified aptamers B060 and B118 resulted in more than 60% neutralization of DENV2 virus. The other modified aptamers resulted in virus neutralization varying between 40% and 58%. This implied that modified aptamers B060 and B118 have the potential to be developed into therapeutics to treat DENV2 infection.
9. Comparison studies for the binding of the modified aptamers (DENV2 rEDIII aptamers) with the other flavivirus envelope proteins (WNV EDIII, TBEV and JEV) shows insignificant binding and is very specific to DENV2 rEDIII.
10. Comparison of the binding of modified aptamer is very high and significant to the DENV2 rEDIII to that of the aptamer obtained from the commercial source.
11. A complete platform for the evaluation of aptamers against DENV2 rEDIII protein is illustrated in FIG. 39.
12. Based on the evaluation, the top three modified aptamer candidates for DENV2 rEDIII protein are B002, B118 and B128, which can be further developed for diagnostic and therapeutic applications. Their sequences are listed in Table 6. These sequences can be further modified for higher affinities.

TABLE 6
Sequences of modified aptamers against DENV2 DIII.

| Product | Adaptamer | |
|---|---|---|
| code | ID | Sequence of variable region |
| Anti-D2ED3- | D2ED3-002 | 5′ T- |
| 01 | | CyA_CfAeT_T_CyAsG_AeT_AeT_ |
| | | GbT_T_GwGbT_T_CyChC_A_Cf-3′ |
| | | (based on modification of |
| | | SEQ ID No. 4) |
| Anti-D2ED3- | D2ED3-118 | 5′-T_AkAlA_T_GwT_GwA_CfGbT_ |
| 02 | | T_CyA_CfAsG_A_CfAlA_GbT_ |
| | | ChC_-3′(based on |
| | | modification of SEQ ID No. 5) |
| Anti-D2ED3- | D2ED3-128 | 5′-GkC_T_GwAeT_A_CfA_ |
| 03 | | CfT_GwAlA_GbT_GbT_T_CyT_ |
| | | GwAeT_T_Gw-3′ (based on |
| | | modification of SEQ ID No. 6) |

1. Backbone nucleotides shown in upper case A: Adenine, G: Guanine, C: Cytosine, T: Thymine
2. Functional groups of side chains shown in lower case: b: Thiophene, e: Glutamic acid, f: Phenylalanine, h: Histidine k: Lysine, l: Leucine, s: Serine, y: Tyrosine, w: Tryptophan
3. Native nucleotides with no side chains shown with an underscore (_)

### REFERENCES

Bhardwaj, S., Holbrook, M., Shope, R. E., Barrett, A. D. and Watowich, S. J. (2001). Biophysical characterization and vector-specific antagonist activity of domain III of the tick-borne flavivirus envelope protein. J Virol 75, 4002-4007.
Bhatt, S., Gething, P. W., Brady, O. J., Messina, J. P., Farlow, A. W., Moyes, C. L., et al. (2013). The global distribution and burden of dengue. Nature.
Bigham, A. W., Buckingham, K. J., Husain, S., Emond, M. J., Bofferding, K. M., Gildersleeve, H., et al. (2011). Host genetic risk factors for West Nile virus infection and disease progression. PloS one 6, e24745.
Chavez, J. H., Silva, J. R., Amarilla, A. A. and Moraes Figueiredo, L. T. (2010). Domain III peptides from flavivirus envelope protein are useful antigens for serologic diagnosis and targets for immunization. Biologicals: journal of the International Association of Biological Standardization 38, 613-618.
Chin, J. F., Chu, J. J. and Ng, M. L. (2007). The envelope glycoprotein domain III of dengue virus serotypes 1 and 2 inhibit virus entry. Microbes Infect 9, 1-6.
Cho, S. J., Woo, H. M., Kim, K. S., Oh, J. W. and Jeong, Y. J. (2011). Novel system for detecting SARS coronavirus nucleocapsid protein using an ssDNA aptamer. J Biosci Bioeng 112, 535-540.
Chu, J. J., Rajamanonmani, R., Li, J., Bhuvanakantham, R., Lescar, J. and Ng, M. L. (2005). Inhibition of West Nile virus entry by using a recombinant domain III from the envelope glycoprotein. J Gen Virol 86, 405-412.
Clyde, K., Kyle, J. L. and Harris, E. (2006). Recent advances in deciphering viral and host determinants of dengue virus replication and pathogenesis. J Virol 80, 11418-11431.
Cohen, C., Forzan, M., Sproat, B., Pantophlet, R., McGowan, I., Burton, D. and James, W. (2008). An aptamer that neutralizes R5 strains of HIV-1 binds to core residues of gp120 in the CCR5 binding site. Virology 381, 46-54.
Crill, W. D. and Roehrig, J. T. (2001). Monoclonal antibodies that bind to domain III of dengue virus E glycoprotein are the most efficient blockers of virus adsorption to Vero cells. J Virol 75, 7769-7773.
Cull, M. G. and Schatz, P. J. (2000). Biotinylation of proteins in vivo and in vitro using small peptide tags. Method Enzymol 326, 430-440.
Dey, A. K., Griffiths, C., Lea, S. M. and James, W. (2005a). Structural characterization of an anti-gp120 RNA aptamer that neutralizes R5 strains of HIV-1. Rna 11, 873-884.
Dey, A. K., Khati, M., Tang, M., Wyatt, R., Lea, S. M. and James, W. (2005b). An aptamer that neutralizes R5 strains of human immunodeficiency virus type 1 blocks gp120-CCR5 interaction. J Virol 79, 13806-13810.
Duan, T., Ferguson, M., Yuan, L., Xu, F. and Li, G. (2009). Human Monoclonal Fab Antibodies Against West Nile Virus and its Neutralizing Activity Analyzed in Vitro and in Vivo. Journal of antivirals & antiretrovirals 1, 36-42.
Fujita, S., Arinaga, K., Fujihara, T., Aki, M. and Kichise, T. (2012). Novel Protein Detection System Using DNA as Constituent Material. Fujitsu Sci Tech 0.148, 237-243.
Giver, L., Bartel, D. P., Zapp, M. L., Green, M. R. and Ellington, A. D. (1993). Selection and Design of High-Affinity Rna Ligands for Hiv-1 Rev. Gene 137, 19-24.
Gopinath, S. C. B., Hayashi, K. and Kumar, P. K. R. (2012). Aptamer That Binds to the gD Protein of Herpes Simplex Virus 1 and Efficiently Inhibits Viral Entry. J Virol 86, 6732-6744.
Gromowski, G. D. and Barrett, A. D. (2007). Characterization of an antigenic site that contains a dominant, type-specific neutralization determinant on the envelope protein domain III (ED3) of dengue 2 virus. Virology 366, 349-360.
James, W. (2007). Aptamers in the virologists' toolkit. J Gen Virol 88, 351-364.
James, W., Moore, M. D., Bunka, D. H. J., Stockley, P. G., Spear, P. G., Cookson, J., et al. (2011). The Development of Aptamers as Candidate Antiviral Agents: Towards a Polyvalent Microbicide. Nucleic Acid Ther 21, A56-A56.
Keefe, A. D., Pai, S. and Ellington, A. (2010). Aptamers as therapeutics. Nat Rev Drug Discov 9, 537-550.
Lisova, O., Hardy, F., Petit, V. and Bedouelle, H. (2007). Mapping to completeness and transplantation of a group-specific, discontinuous, neutralizing epitope in the envelope protein of dengue virus. J Gen Virol 88, 2387-2397.
Liu, Y., Tuleouva, N., Ramanculov, E. and Revzin, A. (2010). Aptamer-Based Electrochemical Biosensor for Interferon Gamma Detection. Anal Chem 82, 8131-8136.
Modis, Y., Ogata, S., Clements, D. and Harrison, S. C. (2004). Structure of the dengue virus envelope protein after membrane fusion. Nature 427, 313-319.
Moore, M. D., Bunka, D. H. J., Forzan, M., Spear, P. G., Stockley, P. G., McGowan, I. and James, W. (2011). Generation of neutralizing aptamers against herpes simplex virus type 2: potential components of multivalent microbicides. J Gen Virol 92, 1493-1499.
Mukhopadhyay, S., Kuhn, R. J. and Rossmann, M. G. (2005). A structural perspective of the flavivirus life cycle. Nat Rev Microbiol 3, 13-22.
Noda, M., Masrinoul, P., Punkum, C., Pipattanaboon, C., Ramasoota, P., Setthapramote, C., et al. (2012). Limited cross-reactivity of mouse monoclonal antibodies against Dengue virus capsid protein among four serotypes. Biologics: targets & therapy 6, 409-416.
Pan, W. H., Craven, R. C., Qiu, Q., Wilson, C. B., Wills, J. W., Golovine, S. and Wang, J. F. (1995). Isolation of Virus-Neutralizing Rnas from a Large Pool of Random Sequences. P Natl Acad Sci USA 92, 11509-11513.
Park, J. H., Jee, M. H., Kwon, O. S., Keum, S. J. and Jang, S. K. (2013). Infectivity of hepatitis C virus correlates with the amount of envelope protein E2: Development of a new aptamer-based assay system suitable for measuring the infectious titer of HCV. Virology 439, 13-22.
Rajamanonmani, R., Nkenfou, C., Clancy, P., Yau, Y. H., Shochat, S. G., Sukupolvi-Petty, S., et al. (2009). On a mouse monoclonal antibody that neutralizes all four dengue virus serotypes. J
Gen Virol 90, 799-809.
Rey, F. A. (2003). Dengue virus envelope glycoprotein structure: New insight into its interactions during viral entry. P Natl Acad Sci USA 100, 6899-6901.
Rey, F. A., Heinz, F. X., Mandl, C., Kunz, C. and Harrison, S. C. (1995). The envelope glycoprotein from tick-borne encephalitis virus at 2 A resolution. Nature 375, 291-298.
Roh, C. and Jo, S. K. (2011). Quantitative and sensitive detection of SARS coronavirus nucleocapsid protein using quantum dots-conjugated RNA aptamer on chip. J Chem Technol Biot 86, 1475-1479.
Saxena, D., Parida, M., Rao, P. V. and Kumar, J. S. (2013). Cloning and expression of an envelope gene of West Nile virus and evaluation of the protein for use in an IgM ELISA. Diagnostic microbiology and infectious disease 75, 396-401.
Silva, M. S. E., Ellis, A., Karaca, K., Minke, J., Nordgren, R., Wu, S. X. and Swayne, D. E. (2013). Domestic goose as a model for West Nile virus vaccine efficacy. Vaccine 31, 1045-1050.
Song, K. M., Lee, S. and Ban, C. (2012). Aptamers and Their Biological Applications. Sensors—Basel 12, 612-631.
Teoh, E. P., Kukkaro, P., Teo, E. W., Lim, A. P., Tan, T. T., Yip, A., et al. (2012). The structural basis for serotype-specific neutralization of dengue virus by a human antibody. Science translational medicine 4, 139ra183.
Tolaymat, N. and Mock, D. M. (1989). Biotin analysis of commercial vitamin and other nutritional supplements. The Journal of nutrition 119, 1357-1360.
Tuerk, C., Macdougal, S. and Gold, L. (1992). Rna Pseudoknots That Inhibit Human-Immunodeficiency-Virus Type-1 Reverse-Transcriptase. P Natl Acad Sci USA 89, 6988-6992.
Wang, P., Yang, Y., Hong, H., Zhang, Y., Cai, W. and Fang, D. (2011). Aptamers as Therapeutics in Cardiovascular Diseases. Curr Med Chem 18, 4169-4174.
Zhang, Z. S., Yan, Y. S., Weng, Y. W., Huang, H. L., Li, S. Q., He, S. and Zhang, J. M. (2007). High-level expression of recombinant dengue virus type 2 envelope domain III protein and induction of neutralizing antibodies in BALB/C mice. J Virol Methods 143, 125-131.
Gromowski G D, Barrett A D. Characterization of an antigenic site that contains a dominant, type-specific neutralization determinant on the envelope protein domain III (ED3) of dengue 2 virus. Virology. 2007; 366(2):349-60. Epub 2007/08/28.
Crill W D, Roehrig J T. Monoclonal antibodies that bind to domain III of dengue virus E glycoprotein are the most efficient blockers of virus adsorption to Vero cells. J Virol. 2001; 75(16):7769-73. Epub 2001/07/20.
Lisova O, Hardy F, Petit V, Bedouelle H. Mapping to completeness and transplantation of a group-specific, discontinuous, neutralizing epitope in the envelope protein of dengue virus. J Gen Virol. 2007; 88(Pt 9):2387-97. Epub 2007/08/19.
Rajamanonmani R, Nkenfou C, Clancy P, Yau Y H, Shochat S G, Sukupolvi-Petty S, et al. On a mouse monoclonal antibody that neutralizes all four dengue virus serotypes. J Gen Virol. 2009; 90(Pt 4):799-809. Epub 2009/03/07.
Rey F A. Dengue virus envelope glycoprotein structure: New insight into its interactions during viral entry. Proceedings of the National Academy of Sciences of the United States of America. 2003; 100(12):6899-901.
Clyde K, Kyle J L, Harris E. Recent advances in deciphering viral and host determinants of dengue virus replication and pathogenesis. J Virol. 2006; 80(23):11418-31. Epub 2006/08/25.
Modis Y, Ogata S, Clements D, Harrison S C. Structure of the dengue virus envelope protein after membrane fusion. Nature. 2004; 427(6972):313-9. Epub 2004/01/23.
Mukhopadhyay S, Kuhn R J, Rossmann M G. A structural perspective of the flavivirus life cycle. Nat Rev Microbiol. 2005; 3(1):13-22. Epub 2004/12/21.
Rey F A, Heinz F X, Mandl C, Kunz C, Harrison S C. The envelope glycoprotein from tick-borne encephalitis virus at 2 A resolution. Nature. 1995; 375(6529):291-8. Epub 1995/05/25.
Bhardwaj S, Holbrook M, Shope R E, Barrett A D, Watowich S J. Biophysical characterization and vector-specific antagonist activity of domain III of the tick-borne flavivirus envelope protein. J Virol. 2001; 75(8):4002-7. Epub 2001/03/27.
Chin J F, Chu J J, Ng M L. The envelope glycoprotein domain III of dengue virus serotypes 1 and 2 inhibit virus entry. Microbes Infect. 2007; 9(1):1-6. Epub 2007/01/02.
Chu J J, Rajamanonmani R, Li J, Bhuvanakantham R, Lescar J, Ng M L. Inhibition of West Nile virus entry by using a recombinant domain III from the envelope glycoprotein. J Gen Virol. 2005; 86(Pt 2):405-12. Epub 2005/01/22.
Zhang Z S, Yan Y S, Weng Y W, Huang H L, Li S Q, He S, et al. High-level expression of recombinant dengue virus type 2 envelope domain III protein and induction of neutralizing antibodies in BALB/C mice. J Virol Methods. 2007; 143(2):125-31. Epub 2007/05/29.
Teoh E P, Kukkaro P, Teo E W, Lim A P, Tan T T, Yip A, et al. The structural basis for serotype-specific neutralization of dengue virus by a human antibody. Science translational medicine. 2012; 4(139):139ra83. Epub 2012/06/23.
Noda M, Masrinoul P, Punkum C, Pipattanaboon C, Ramasoota P, Setthapramote C, et al. Limited cross-reactivity of mouse monoclonal antibodies against Dengue virus capsid protein among four serotypes. Biologics: targets & therapy. 2012; 6:409-16. Epub 2012/12/05.
Bhatt S, Gething P W, Brady O J, Messina J P, Farlow A W, Moyes C L, et al. The global distribution and burden of dengue. Nature. 2013. Epub 2013/04/09.
Tuerk C, Macdougal S, Gold L. Rna Pseudoknots That Inhibit Human-Immunodeficiency-Virus Type-1 Reverse-Transcriptase. P Natl Acad Sci USA. 1992; 89(15):6988-92.
Giver L, Bartel D P, Zapp M L, Green M R, Ellington A D. Selection and Design of High-Affinity Rna Ligands for Hiv-1 Rev. Gene. 1993; 137(1):19-24.
Cohen C, Forzan M, Sproat B, Pantophlet R, McGowan I, Burton D, et al. An aptamer that neutralizes R5 strains of HIV-1 binds to core residues of gp120 in the CCR5 binding site. Virology. 2008; 381(1):46-54.
Dey A K, Khati. M, Tang M, Wyatt R, Lea S M, James W. An aptamer that neutralizes R5 strains of human immunodeficiency virus type 1 blocks gp120-CCR5 interaction. J Virol. 2005; 79(21): 13806-10.
Dey A K, Griffiths C, Lea S M, James W. Structural characterization of an anti-gp120 RNA aptamer that neutralizes R5 strains of HIV-1. Rna. 2005; 11(6):873-84.
Pan W H, Craven R C, Qiu Q, Wilson C B, Wills J W, Golovine S, et al. Isolation of Virus-Neutralizing Rnas from a Large Pool of Random Sequences. P Natl Acad Sci USA. 1995; 92(25): 11509-13.
Cho S J, Woo H M, Kim K S, Oh J W, Jeong Y J. Novel system for detecting SARS coronavirus nucleocapsid protein using an ssDNA aptamer. J Biosci Bioeng. 2011; 112(6):535-40.
Park J H, Jee M H, Kwon O S, Keum S J, Jang S K. Infectivity of hepatitis C virus correlates with the amount of envelope protein E2: Development of a new aptamer-based assay system suitable for measuring the infectious titer of HCV. Virology. 2013; 439(1):13-22. Epub 2013/03/15.
Wang P, Yang Y, Hong H, Zhang Y, Cai W, Fang D. Aptamers as Therapeutics in Cardiovascular Diseases. Curr Med Chem. 2011; 18(27):4169-74.
Keefe A D, Pai S, Ellington A. Aptamers as therapeutics. Nat Rev Drug Discov. 2010; 9(7):537-50.
Roh C, Jo S K. Quantitative and sensitive detection of SARS coronavirus nucleocapsid protein using quantum dots-conjugated RNA aptamer on chip. J Chem Technol Biot. 2011; 86(12): 1475-9.
Song K M, Lee S, Ban C. Aptamers and Their Biological Applications. Sensors-Basel. 2012; 12(1):612-31.
Liu Y, Tuleouva N, Ramanculov E, Revzin A. Aptamer-Based Electrochemical Biosensor for Interferon Gamma Detection. Anal Chem. 2010; 82(19):8131-6.
Gopinath S C B, Hayashi K, Kumar P K R. Aptamer That Binds to the gD Protein of Herpes Simplex Virus 1 and Efficiently Inhibits Viral Entry. J Virol. 2012; 86(12):6732-44.
James W, Moore M D, Bunka D H J, Stockley P G, Spear P G, Cookson J, et al. The Development of Aptamers as Candidate Antiviral Agents: Towards a Polyvalent Microbicide. Nucleic Acid Ther. 2011; 21(5):A56-A.
Moore M D, Bunka D H J, Forzan M, Spear P G, Stockley P G, McGowan I, et al. Generation of neutralizing aptamers against herpes simplex virus type 2: potential components of multivalent microbicides. J Gen Virol. 2011; 92:1493-9.
James W. Aptamers in the virologists' toolkit. J Gen Virol. 2007; 88:351-64.
Fujita S, Arinaga K, Fujihara T, Aki M, Kichise T. Novel Protein Detection System Using DNA as Constituent. Material. Fujitsu Sci Tech J. 2012; 48(2):237-43.
Kaur H, Li J J, Bay B H, Yung L Y. Investigating the antiproliferative activity of high affinity DNA aptamer on cancer cells. PloS one. 2013; 8(1):e50964. Epub 2013/01/24.
Peng C G, Damha M J. G-quadruplex induced stabilization by 2′-deoxy-2′-fluoro-D-arabinonucleic acids (2′F-ANA). Nucleic acids research. 2007; 35(15):4977-88. Epub 2007/07/20.
Mallikaratchy P R, Ruggiero A, Gardner J R, Kuryavyi V, Maguire W F, Heaney M L, et al. A multivalent DNA aptamer specific for the B-cell receptor on human lymphoma and leukemia. Nucleic acids research. 2011; 39(6):2458-69. Epub 2010/10/30.

All references herein mentioned are hereby incorporated by reference.

1. A nucleic acid aptamer comprising a DNA molecule that binds specifically to a flavivirus structural protein or a flavivirus non-structural protein.

2. The aptamer according to claim 1, wherein the flavivirus is selected from the group consisting of West Nile virus, Dengue virus, yellow fever virus, Japanese encephalitis, and tick-borne encephalitis virus.

3. The aptamer according to claim 1, wherein the aptamer binds specifically to a West Nile virus envelope protein.

4. The aptamer according to claim 3, wherein the aptamer binds specifically to Domain III region of the West Nile virus envelope protein.

5. The aptamer according to claim 1, wherein the DNA molecule comprises amino acid side chains.

6. The aptamer according to claim 3, wherein the DNA molecule comprises amino acid side chains and wherein the DNA molecule comprises a sequence selected from the group consisting of:
(a) 5′-A_CfGkC_T_GwChC_A_CfAlA_GbT_ChC_T_GwGbT_T_CyChC_T_Gw-3′ (based on modification of SEQ ID No. 1) or its complement;
(b) 5′-ChC_T_CyChC_AlA_A_CfAeT_GbT_AsG_AsG_T_CyT_CyA_CfAeT-3′ (based on modification of SEQ ID No. 2) or its complement; and
(c) 5′-ChC_AlA_AeT_T_GwChC_GkC_AsG_A_CfT_CyGbT_T_GwT_GwAlA_-3′ (based on modification of SEQ ID No. 3) or its complement,
wherein functional groups of side chains are indicated in lowercase (b: Thiophene, e: Glutamic acid, f: Phenylalanine, h: Histidine, k: Lysine, l: Leucine, s: Serine, y: Tyrosine, w: Tryptophan) and unmodified native nucleotides are indicated with an underscore (_).

7. The aptamer according to claim 1, wherein the aptamer binds specifically to a Dengue virus envelope protein.

8. The aptamer according to claim 7, wherein the aptamer binds specifically to Domain III region of the Dengue virus envelope protein.

9. The aptamer according to claim 7, wherein the DNA molecule comprises amino acid side chains.

10. The aptamer according to claim 9, wherein the DNA molecule comprises a sequence selected from the group consisting of:
(a) 5′ T-CyA_CfAeT_T_CyAsG_AeT_AeT_GbT_T_GwGbT_T_CyChC_A_Cf-3′ (based on modification of SEQ ID No. 4) or its complement;
(b) 5′-T_AkAlA_T_GwT_GwA_CfGbT_T_CyA_CfAsG_A_CfAlA_GbT_ChC_-3′ (based on modification of SEQ ID No. 5) or its complement; and
(c) 5′-GkC_T_GwAeT_A_CfA_CfT_GwAlA_GbT_GbT_T_CyT_GwAeT_T_Gw-3′ (based on modification of SEQ ID No. 6) or its complement
wherein functional groups of side chains are indicated in lowercase (b: Thiophene, e: Glutamic acid, f: Phenylalanine, h: Histidine, k: Lysine, l: Leucine, s: Serine, y: Tyrosine, w: Tryptophan) and unmodified native nucleotides are indicated with an underscore (_).

11. The aptamer according to claim 1, wherein the DNA molecule further comprises a detectable moiety.

12. The aptamer according to claim 11, wherein the detectable moiety is selected from the group consisting of biotin, enzymes, chromophores, fluorescent molecules, chemiluminescent molecules, phosphorescent molecules, coloured particles, and luminescent molecules.

13. The aptamer according to claim 12, wherein the detectable moiety is biotin.

14. The aptamer according to claim 1, further comprising a drug of interest, wherein the binding of the DNA molecule to a flavivirus structural protein or a flavivirus non-structural protein targets the drug of interest to its intended site of action and/or releases the drug of interest from the aptamer.

15. The aptamer according to claim 14, wherein the drug is selected from the group consisting of a pharmaceutical compound, a nucleotide, an antigen, a steroid, a vitamin, a hapten, a metabolite, a peptide, a protein, a peptidomimetic compound, an imaging agent, an anti-inflammatory agent, a cytokine, and an immunoglobulin molecule or fragment thereof.

16. The aptamer according to claim 1 for use in diagnosis of a flavivirus infection in a patient.

17. The aptamer according to claim 1 for use in therapy.

18. An immunogenic composition or vaccine comprising an aptamer according to claim 1.

19. A composition comprising an aptamer according to claim 1 and an excipient or carrier.

20. A kit comprising an aptamer according to claim 1 and a carrier.

21. A method for diagnosing or detecting a flavivirus infection in a patient, the method comprising:
(a) obtaining a biological sample from a patient;
(b) contacting the biological sample with an aptamer according to any one of claims 1 to 15;
(c) detecting the formation of the binding complex between the aptamer and a flavivirus structural protein and/or a flavivirus non-structural protein,
wherein the presence of the binding complex indicates that the patient has a flavivirus infection.

22. The method of claim 21, wherein the biological sample is a blood sample, serum, plasma, saliva or urine.

23. A method for treating or inducing an immune response to a flavivirus infection in a patient, the method comprising administering to the patient a therapeutically effective dose of the composition or vaccine according to claim 18.

24. Use of an aptamer according to claim 1 for treating a flavivirus infection in a patient.

25. Use of an aptamer according to claim 1 in the manufacture of a medicament for treating or preventing a flavivirus infection in a patient.

26. The aptamer according to claim 1, for use in treating or preventing a flavivirus infection in a patient.
